SEQUENCE LISTING (20 sequences)

SEQ ID NO: 1 - DNA, 1920 bp, Homo sapiens
  source 1..1920: mol_type = other DNA
atgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 2 - protein, 639 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 3 - DNA, 1947 bp, unidentified
  source 1..1947: mol_type = other DNA; note = RABBIT
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagcctgatgacgttgtttacaaacaatatgagagttatacagtaaccatcacctgccctttcacatatgcgactaggcaactaaagaagtccttttacaaggtggaagacggggaacttgtactcatcattgattccagcagtaaggaggcaaaggaccccaggtataagggcagaataacgttgcagatccaaagtaccacagcaaaagaattcacagtcaccatcaagcatttgcagctcaatgatgctgggcagtatgtctgccagagtggaagcgaccccactgctgaagaacagaacgttgacctccgactgctaactcctggtctgctctatggaaacctggggggctcggtgacctttgaatgtgccctggactctgaagacgcaaacgcggtagcatccttgcgccaggttaggggtggcaatgtggtcattgacagccaggggacaatagatccagccttcgagggcaggatcctgttcaccaaggctgagaacggccacttcagtgtagtgatcgcaggcctgaggaaggaagacacagggaactatctgtgcggagtccagtccaatggtcagtctggggatgggcccacccagcttcggcaactcttcgtcaatgaagagatcgacgtgtcccgcagcccccctgtgttgaagggctttccaggaggctccgtgaccatacgctgcccctacaacccgaagagaagcgacagccacctgcagctgtatctctgggaagggagtcaaacccgccatctgctggtggacagcggcgaggggctggttcagaaagactacacaggcaggctggccctgttcgaagagcctggcaatggcaccttctcagtcgtcctcaaccagctcactgccgaggatgaaggcttctactggtgtgtcagcgatgacgatgagtccctgacgacttcggtgaagctccagatcgttgacggagaaccaagccccacgatcgacaagttcactgctgtgcagggagagcctgttgagatcacctgccacttcccatgcaaatacttctcctccgagaagtactggtgcaagtggaatgaccatggctgcgaggacctgcccactaagctcagctccagcggcgaccttgtgaaatgcaacaacaacctggtcctcaccctgaccttggactcggtcagcgaagatgacgagggctggtactggtgtggcgcgaaagacgggcacgagtttgaagaggttgcggccgtcagggtggagctgacagagccagccaaggtagctgtcgagccagccaaggtacctgtcgacccagccaaggcagcccccgcgcctgctgaggagaaggccaaggcgcggtgcccagtgcccaggagaaggcagtggtacccattgtcaaggaagctgagaacaagttgtccagaacctcggctccttgcggaggaggtagcagtgcagagtgcggaagacccagccagtgggagcagagcgtctgtggatgccagcagtgcttcgggacaaagcgggagtgccaaaaggtga

SEQ ID NO: 4 - protein, 648 aa, unidentified
  source 1..648: note = RABBIT
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKARCPVPRRRQWYPLSRKLRTSCPEPRLLAEEVAVQSAEDPASGSRASVDASSASGQSGSAKR

SEQ ID NO: 5 - DNA, 1935 bp, synthetic construct
  misc_feature 1..1935: note = CHIMERA (R/HpIgR)
  source 1..1935: mol_type = other DNA
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 6 - protein, 644 aa, synthetic construct
  REGION 1..644: note = CHIMERA (R/HpIgR)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPELLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 7 - DNA, 411 bp, synthetic construct
  misc_feature 1..411: note = CHIMERA (R/HpIgR)
  source 1..411: mol_type = other DNA
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagag

SEQ ID NO: 8 - protein, 137 aa, synthetic construct
  REGION 1..137: note = CHIMERA (R/HpIgR)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPE

SEQ ID NO: 9 - DNA, 1524 bp, synthetic construct
  misc_feature 1..1524: note = CHIMERA (R/HpIgR)
  source 1..1524: mol_type = other DNA
ctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtga

SEQ ID NO: 10 - protein, 507 aa, synthetic construct
  REGION 1..507: note = CHIMERA (R/HpIgR)
LLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRR

SEQ ID NO: 11 - DNA, 2031 bp, synthetic construct
  misc_feature 1..2031: note = HUMAN pIgR-CD4 cyto (HpIgR-cyto)
  source 1..2031: mol_type = other DNA
atgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 12 - protein, 676 aa, synthetic construct
  REGION 1..676: note = HUMAN pIgR-CD4 cyto (HpIgR-cyto)
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

SEQ ID NO: 13 - DNA, 2058 bp, synthetic construct
  misc_feature 1..2058: note = RABBIT pIgR-CD4 cyto (RpIgR-cyto)
  source 1..2058: mol_type = other DNA
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagcctgatgacgttgtttacaaacaatatgagagttatacagtaaccatcacctgccctttcacatatgcgactaggcaactaaagaagtccttttacaaggtggaagacggggaacttgtactcatcattgattccagcagtaaggaggcaaaggaccccaggtataagggcagaataacgttgcagatccaaagtaccacagcaaaagaattcacagtcaccatcaagcatttgcagctcaatgatgctgggcagtatgtctgccagagtggaagcgaccccactgctgaagaacagaacgttgacctccgactgctaactcctggtctgctctatggaaacctggggggctcggtgacctttgaatgtgccctggactctgaagacgcaaacgcggtagcatccttgcgccaggttaggggtggcaatgtggtcattgacagccaggggacaatagatccagccttcgagggcaggatcctgttcaccaaggctgagaacggccacttcagtgtagtgatcgcaggcctgaggaaggaagacacagggaactatctgtgcggagtccagtccaatggtcagtctggggatgggcccacccagcttcggcaactcttcgtcaatgaagagatcgacgtgtcccgcagcccccctgtgttgaagggctttccaggaggctccgtgaccatacgctgcccctacaacccgaagagaagcgacagccacctgcagctgtatctctgggaagggagtcaaacccgccatctgctggtggacagcggcgaggggctggttcagaaagactacacaggcaggctggccctgttcgaagagcctggcaatggcaccttctcagtcgtcctcaaccagctcactgccgaggatgaaggcttctactggtgtgtcagcgatgacgatgagtccctgacgacttcggtgaagctccagatcgttgacggagaaccaagccccacgatcgacaagttcactgctgtgcagggagagcctgttgagatcacctgccacttcccatgcaaatacttctcctccgagaagtactggtgcaagtggaatgaccatggctgcgaggacctgcccactaagctcagctccagcggcgaccttgtgaaatgcaacaacaacctggtcctcaccctgaccttggactcggtcagcgaagatgacgagggctggtactggtgtggcgcgaaagacgggcacgagtttgaagaggttgcggccgtcagggtggagctgacagagccagccaaggtagctgtcgagccagccaaggtacctgtcgacccagccaaggcagcccccgcgcctgctgaggagaaggccaaggcgcggtgcccagtgcccaggagaaggcagtggtacccattgtcaaggaagctgagaacaagttgtccagaacctcggctccttgcggaggaggtagcagtgcagagtgcggaagacccagccagtgggagcagagcgtctgtggatgccagcagtgcttcgggacaaagcgggagtgccaaaaggtgccggcaccgaaggcgccaagcagagcggatgtctcagatcaagagactcctcagtgagaagaagacctgccagtgccctcaccggtttcagaagacatgtagccccatttga

SEQ ID NO: 14 - protein, 685 aa, synthetic construct
  REGION 1..685: note = RABBIT pIgR-CD4 cyto (RpIgR-cyto)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPEPDDVVYKQYESYTVTITCPFTYATRQLKKSFYKVEDGELVLIIDSSSKEAKDPRYKGRITLQIQSTTAKEFTVTIKHLQLNDAGQYVCQSGSDPTAEEQNVDLRLLTPGLLYGNLGGSVTFECALDSEDANAVASLRQVRGGNVVIDSQGTIDPAFEGRILFTKAENGHFSVVIAGLRKEDTGNYLCGVQSNGQSGDGPTQLRQLFVNEEIDVSRSPPVLKGFPGGSVTIRCPYNPKRSDSHLQLYLWEGSQTRHLLVDSGEGLVQKDYTGRLALFEEPGNGTFSVVLNQLTAEDEGFYWCVSDDDESLTTSVKLQIVDGEPSPTIDKFTAVQGEPVEITCHFPCKYFSSEKYWCKWNDHGCEDLPTKLSSSGDLVKCNNNLVLTLTLDSVSEDDEGWYWCGAKDGHEFEEVAAVRVELTEPAKVAVEPAKVPVDPAKAAPAPAEEKAKARCPVPRRRQWYPLSRKLRTSCPEPRLLAEEVAVQSAEDPASGSRASVDASSASGQSGSAKRCRHRRRQAERMSQIKRLLSEKKTCQCPHRFQKTCSPI

SEQ ID NO: 15 - DNA, 2046 bp, synthetic construct
  misc_feature 1..2046: note = CHIMERA-CD4 cyto (R/HpIgR-cyto)
  source 1..2046: mol_type = other DNA
atggctctcttcttgctcacctgcctgctggctgtcttttcagcggccacggcacaaagctccttattgggtcccagctccatatttggtcccggggaggtgaatgttttggaaggcgactcggtgtccatcacatgctactacccaacaacctccgtcacccggcacagccggaagttctggtgccgggaagaggagagcggccgctgcgtgacgcttgcctcgaccggctacacgtcccaggaatactccgggagaggcaagctcaccgacttccctgataaaggggagtttgtggtgactgttgaccaactcacccagaacgactcagggagctacaagtgtggcgtgggagtcaacggccgtggcctggacttcggtgtcaacgtgctggtcagccagaagccagagctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 16 - protein, 681 aa, synthetic construct
  REGION 1..681: note = CHIMERA-CD4 cyto (R/HpIgR-cyto)
MALFLLTCLLAVFSAATAQSSLLGPSSIFGPGEVNVLEGDSVSITCYYPTTSVTRHSRKFWCREEESGRCVTLASTGYTSQEYSGRGKLTDFPDKGEFVVTVDQLTQNDSGSYKCGVGVNGRGLDFGVNVLVSQKPELLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

SEQ ID NO: 17 - DNA, 411 bp, synthetic construct; identical to SEQ ID NO: 7
  misc_feature 1..411: note = CHIMERA-CD4 cyto (R/HpIgR-cyto)
  source 1..411: mol_type = other DNA

SEQ ID NO: 18 - protein, 137 aa, synthetic construct; identical to SEQ ID NO: 8
  REGION 1..137: note = CHIMERA-CD4 cyto (R/HpIgR-cyto)

SEQ ID NO: 19 - DNA, 1635 bp, synthetic construct
  misc_feature 1..1635: note = CHIMERA-CD4 cyto (R/HpIgR-cyto)
  source 1..1635: mol_type = other DNA
ctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaggctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagaaggtgccggcaccgaaggcgccaagcagagcggatgtctcagatcaagagactcctcagtgagaagaagacctgccagtgccctcaccggtttcagaagacatgtagccccatttga

SEQ ID NO: 20 - protein, 544 aa, synthetic construct
  REGION 1..544: note = CHIMERA-CD4 cyto (R/HpIgR-cyto)
LLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRRCRHRRRQAERMSQIKRLLSEKKTCQCPHRFQKTCSPI

(19) United States
(12) Patent Application Publication
(10) Pub. No.: US 2025/0122262 A1
(43) Pub. Date: Apr. 17, 2025

(54) DIAGNOSTIC, PROGNOSTIC, THERAPEUTIC AND SCREENING PROTOCOLS

(71) Applicant: The MacFarlane Burnet Institute for Medical Research and Public Health Ltd, Melbourne (AU)

(72) Inventors: David Andrew Anderson, Melbourne (AU); Mary Louise Garcia, Melbourne (AU); Nadine Carmel Barnes, Melbourne (AU); Khayriyyah Mohd Hanafiah, Melbourne (AU); Alan Lee Landay, Melbourne (AU)

(73) Assignee: The MacFarlane Burnet Institute for Medical Research and Public Health Ltd, Melbourne (AU)

(21) Appl. No.: 18/938,734

(22) Filed: Nov. 6, 2024

Related U.S. Application Data
(63) Continuation of application No. 16/271,588, filed on Feb. 8, 2019, now Pat. No. 12,168,682.
Application No. 16/271,588 is a continuation of application No. 14/441,606, filed on May 8, 2015, now abandoned.

(30) Foreign Application Priority Data
Nov. 8, 2012 (AU) 2012904887

(51) Int. Cl. C07K 14/705; G01N 33/569; G01N 33/68

(52) U.S. Cl. CPC C07K 14/70503; G01N 33/56983; G01N 33/6854; C07K 2319/00; G01N 2333/08; G01N 2333/10; G01N 2333/16; G01N 2333/70535; G01N 2469/20; G01N 2800/06

(57) ABSTRACT

The specification describes an antibody capture process comprising (i) obtaining a biological sample comprising antibodies, (ii) contacting the biological sample with recombinant pIgR or a dIgA-binding variant, wherein the pIgR or variant binds dIgA and forms a pIgR-dIgA complex. The process may further comprise (iii) directly or indirectly assessing the level of the pIgR-dIgA complex or the level of a complex between pIgR-dIgA and an antigen of interest. There is also an antibody capture process for determining gut wall integrity in a test subject, wherein the level or ratio of SIgA to dIgA is compared to a corresponding level or ratio from a control subject. The specification provides kits embodying the process and recombinant pIgR when used for, or for use, in capturing or detecting dIgA and/or IgM.

### CROSS-REFERENCE TO RELATED APPLICATIONS

The present application is a continuation of U.S. Ser. No. 16/271,588, filed 8 Feb. 2019, now U.S. Pat. No. 12,168,682, issued Dec. 17, 2024, which is a continuation of U.S. Ser. No. 14/441,606, filed 8 May 2015, which is a 35 U.S.C. § 371 U.S. National phase application of International Patent Application No. PCT/AU2013/001291, filed 8 Nov. 2013, which claims priority from Australian Provisional Patent Application No. 2012904887 filed 8 Nov. 2012, the disclosures of all of the above applications are incorporated by reference herein in their entireties.

### SEQUENCE LISTING

This application incorporates by reference in its entirety the Sequence Listing prepared Nov. 6, 2024 and entitled “999917-516985US02_SL.xml”, which is 46,058 bytes in size, filed electronically herewith.

### FIELD

The present specification relates generally to the fields of diagnostic, prognostic and therapeutic protocols with respect to infectious agents or other conditions associated with immune activation and particularly mucosal immune activation. More particularly, the specification relates to the use of antibodies as biomarkers for immune activation and/or for diagnosis, prognosis and treatment of conditions associated with immune activation. The present protocols are proposed for ready translation into both laboratory and point of care formats to reach target populations worldwide.

### BACKGROUND

Bibliographic details references referred to in this specification are listed at the end of the specification.

The reference to any prior art is not and should not be taken as an acknowledgment or any form of suggestion that this prior art forms part of the common general knowledge in any country.

The detection of specific antibody (immunoglobulin (Ig)) classes is recognized as an important step in diagnostic and research methods for human and animal diseases. For example, detection of antigen-specific IgM-class antibodies is widely used as a diagnostic test for infection with viruses such as hepatitis A virus, hepatitis E virus, West Nile virus, dengue viruses, measles virus, rubella virus; and for infection with bacteria such as syphilis (Treponema pallidum), because IgM class antibodies are typically made in the body of an infected host during the acute phase of infection and are detectable for only a few months.

Conversely, IgG-class antibodies commonly persist for life and may indicate either current or past infection with a specific agent. For chronic infections such as the human immunodeficiency virus (HIV) where patients do not spontaneously clear the virus, detection of IgG-class antibodies is diagnostic for infection, whereas for others such as hepatitis C virus (HCV) where a proportion of patients do clear the virus either spontaneously or following treatment, the detection of antigen-specific IgG is not diagnostic of current or ongoing infection. IgG-class antibodies are also primarily responsible for antibody-mediated immunity within the plasma compartment of the body.

IgA-class antibodies have also been used to aid diagnosis of infections including hepatitis E virus, hepatitis A virus, and dengue viruses, as well as in the study of vaccines and immunity to infections. IgA is attractive for diagnostic purposes, because it is predominantly made during the acute phase of infection, and high levels of antigen-specific IgA can provide a marker of current infection, with or without the concurrent detection of IgM. In addition, because IgA is the predominant antibody class that is secreted at mucosal epithelial surfaces, its presence is considered as a marker of mucosal immunity. The role of different IgA structural forms as biomarkers for infection, such as specifically dIgA, is not understood. The role of SIgA in infection and antigens that engender SIgA responses have not been explored nor have diagnostic and prognostic protocols been developed that are designed to rapidly and conveniently assess these responses in patient sera.

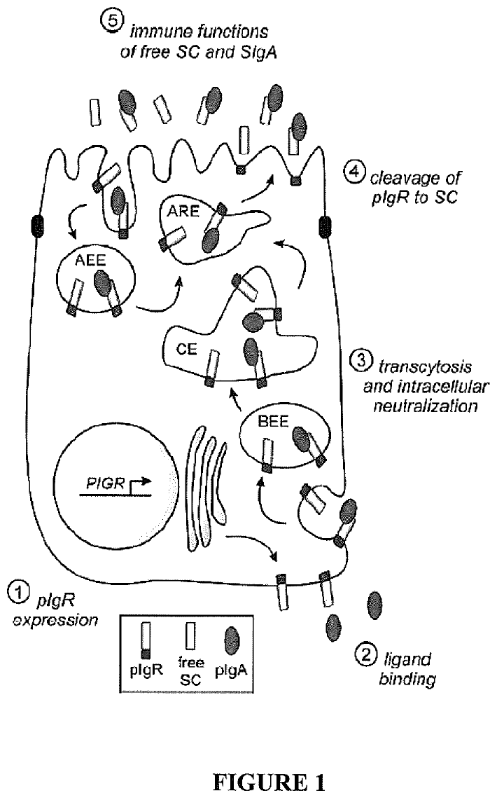

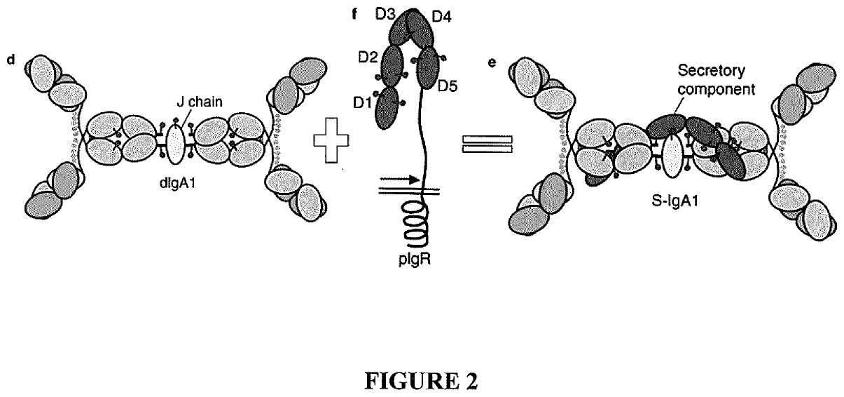

In most animals, IgA is synthesized almost exclusively as dimeric or higher polymeric forms, here described collectively as dIgA, which are able to interact with the polymeric Ig receptor (pIgR). This interaction results in secretion of large amounts of secretory IgA (SIgA) into the lumen of epithelial tissues (see FIGS. 1 and 2). However, in humans and higher primates the dIgA is only a minor fraction of the total IgA, with monomeric IgA (mIgA) representing around 90% of the total IgA, and dimeric or higher polymeric forms of IgA representing around 10% of the total IgA.

Detection of IgA, IgM, IgG and other antibody classes or isotypes is usually performed using antibody reagents prepared in another species, for example rabbit antibodies specific for human IgM, or mouse monoclonal antibodies specific for human IgA, or monoclonal antibodies specific for individual antibody subclasses such as IgA1, IgA2 or IgG1, IgG2a, IgG2b, IgG3, IgG4. Antibody based capture assay are associated with levels of non-specific binding which is minimised through optimisation protocols.

There is a need for improved serological protocols for monitoring infections associated with mucosal surfaces and mucosal immune responses in a subject, and for agents that can be used to assess dimeric or polymeric antibody production and or for dimeric or polymeric antibody purification.

### SUMMARY OF EMBODIMENTS

In one embodiment, therefore, the specification provides an antibody capture process comprising (i) obtaining a biological sample comprising antibodies, (ii) contacting the biological sample with recombinant pIgR or a dIgA-binding variant, wherein the pIgR or variant binds dIgA and forms a pIgR-dIgA complex. Once the complex has been formed, the complex may be quantified. In some embodiments, dIgA may be released from the pIgR and further processed. In one embodiment, the process is employed for the purification of dIgA antibodies as an alternative to existing processes which employ jacalin agarose. Detection of complexes uses routine methods and agents known in the art such as ELISA or other immunoassay based methods.

In one embodiment, the process further comprises (iii) directly or indirectly assessing the level of the pIgR-sIgA complex or the level of a complex between pIgR-dIgA and an antigen of interest.

In one embodiment, the pIgR or a dIgA-binding variant binds dIgA and substantially fails to bind IgM or wherein the pIgR or variant binds dIgA and IgM. Processes for detecting antigen specific IgM and dIgA may be used in conjunction with tests for total IgA, IgG and other individual isotypes, subclasses and structural forms and combinations of two or more of these.

In one embodiment, the biological sample is a blood or serum sample. Alternative biological samples include samples comprising cells expressing dIgA. Thus, in some embodiments, the process may be used to detect individual B-cells that express dIgA in the screening or isolation of immortalized B-cells.

In another embodiment, the biological sample is obtained from a subject. The subject is human or primate in some embodiments, and a mammalian or avian animal species other than a primate or human species in another embodiment. Various forms of recombinant pIgR are selected based on the target antibody of interest and species from which the antibody is derived.

As illustrated herein in one embodiment, the pIgR is recombinant HpIgA or RpIgR. Conveniently, in one embodiment, the recombinant pIgR or dIgA-binding variant has the transmembrane domain and/or the cytoplasmic domain deleted. In other embodiments, the recombinant pIgR comprises a heterologous detection or binding domain. In another embodiment, the recombinant pIgR or IgA-binding variant is recombinantly produced in a glycan deficient cell, such as a CHO cell.

In exemplary embodiments of the process, the recombinant pIgR is bound to a solid support.

In one embodiment, the biological sample is depleted of IgM or dIgA antibodies prior to use in the process. This allows the pIgR which binds to IgM and dIgA to be employed in assays to specifically detect dIgA. Similarly, depletion of dIgA (using, for example, R/HpIgR) facilitates the use of HpIgR in assays to specifically detect IgM. In some embodiments, IgG is depleted prior to sample use in order to reduce competition with dIgA. However, as competing antibodies are washed away in the present antibody/isotype capture formats, lack of such competition is an advantage of the process.

Any antigen of interest may be employed and in one non-limiting embodiments, the antigen of interest is an antigen of an infectious agent or an antigen associated with a condition of a subject that affects a mucosal surface or associated tissues. For example, infective agents include HIV, leprosy, syphilis, hepatitis, dengue virus, measles and rubella.

In one embodiment, the process further comprises contacting the biological sample with an anti-SC binding agent or anti-SC antibody wherein the anti-SC binding agent or anti-SC antibody binds SIgA and forms an SIgA-binding agent/antibody complex. In another embodiment, the process further comprises contacting a sample comprising the pIgR-dIgA complex with a denaturing solution to remove any SIgA from the complex and measuring the ratio of SIgA and dIgA in the biological sample.

In another aspect, the specification enables an antibody capture process for determining gut wall integrity in a test subject, the process comprising (i) obtaining a biological sample comprising antibodies from the test subject, (ii) contacting the biological sample with recombinant pIgR or a dIgA-binding variant, wherein the pIgR or variant binds dIgA and forms a pIgR-dIgA complex, and (iii) contacting the biological sample with a specific anti-SIgA binding agent or anti-SC binding agent/antibody wherein the anti-SIgA binding agent or anti-SC binding agent/antibody binds SIgA and forms an SIgA-binding agent/antibody complex, and (iv) measuring and comparing the level of the complex formed in (ii) with the level of the complex formed in (iii), wherein the ratio of SIgA to dIgA is compared to a corresponding level or ratio from a control subject and provides a measure of gut integrity/leakage.

In one embodiment of the process, the level or ratio of SIgA2/dIgA2 and/or SIgA1/dIgA1 are determined.

In another embodiment, the antibody capture process comprises (i) obtaining a biological sample comprising antibodies, (ii) contacting the biological sample with recombinant pIgR or a dIgA or IgM-binding variant, wherein the pIgR or variant binds IgM and/or IgA and forms a pIgR-IgM and/or pIgR-dIgA complex.

Alternatively, the antibody capture process may comprise (i) obtaining a biological sample comprising antibodies, (ii) contacting the biological sample with recombinant pIgR wherein the pIgR or variant binds IgM and forms a pIgR-IgM complex.

In another illustrated embodiment, a process is enabled for detecting the presence of antigen-specific dIgA in a subject, the process comprising (i) obtaining a biological sample comprising antibodies from a subject, (ii) contacting the sample with R/HpIgR and antigen and (iii) measuring the level of antigen-specific dIgA.

In another embodiment, a process is described for detecting the presence of antigen-specific IgM in a subject, the process comprising (i) obtaining a biological sample comprising antibodies from a subject, (ii) contacting the sample with a HpIgR and antigen and (iii) measuring the level of antigen-specific IgM.

In one embodiment, the process is for detecting the presence of antigen-specific IgM and dIgA in a subject, the process comprising (i) obtaining a biological sample comprising antibodies from a subject, (ii) contacting the sample with HpIgR and R/HpIgR and antigen and (iii) measuring the level of antigen-specific IgM and antigen specific dIgA.

In yet another aspect, the present specification enables a kit for assessing immune status in a subject, the kit comprising, (a) an immunographic device comprising a porous membrane operably connected to a sample portion, a test portion, and optionally a control portion; and further comprising a sucker portion, portion comprising a recombinant pIgR molecule or dIgA-binding variant thereof, a portion comprising an antigen of interest and optionally a conjugate portion; and b) instructions for using the immunographic device to detect the presence of antigen specific dIgA antibody in a biological sample obtained from the subject.

As described herein in relation to the process, in some embodiments of the kit, the pIgR is HpIgR and/or R/HpIgR. In some embodiments, the recombinant pIgR or dIgA-binding variant has the transmembrane domain and/or the cytoplasmic domain deleted. In one embodiment, the recombinant pIgR comprises a heterologous detection or binding domain. In yet another embodiment, the recombinant pIgR or IgA-binding variant is recombinantly produced in a glycan deficient cell.

In one embodiment, the recombinant pIgR is bound to a solid support.

In one embodiment, the biological sample is depleted of IgM or dIgA antibodies prior to use in the process. The kits and reagents contained therein of the present invention are for use ex vivo.

Any antigen may be employed however in one embodiment, the antigen of interest is an antigen of an infectious agent or an antigen associated with a condition of a subject that affects a mucosal surface or associated tissues. Illustrative infectious agents are selected from HIV, leprosy, syphilis, hepatitis, dengue virus, measles and rubella. The detection of elevated levels of antigen specific dIgA recombinant pIgR relative to control levels facilitates diagnosis and the selection and treatment options. In some embodiments, a method of treatment is contemplated comprising requesting a test for antigen-specific dIgA levels and administered treatment to the diagnosed subject if the test is positive for an infection or condition.

In another embodiment, the kit further comprises an anti-SIgA binding agent/antibody or anti-SC antibody, wherein the anti-SIgA binding agent/antibody or anti-SC antibody binds SIgA and forms an SIgA-binding agent/antibody complex.

In another aspect, recombinant pIgR is provided which is suitable for use in capturing or detecting dIgA and/or IgM. Illustrative recombinant pIgRs include R/HpIgR or HpIgR or a dIgA and/or IgM binding variant of R/HpIgR or HpIgR. Illustrative amino acid and nucleotide sequences are set out in SEQ ID NO:1 to 20, bearing in mind that some of these sequence encode or provide a CD4 cytoplasmic domain which is entirely optional and may be deleted, modified, supplemented or replaced with other binding or detection molecules known in the art. Once the subject invention is contemplated, useful variants of recombinant pIgR will be apparent to the skilled person and readily made and tested.

### DETAILED DESCRIPTION OF THE FIGURES SUPPORTING AND DESCRIBING THE SUBJECT PROCESSES AND KITS

If figures contain colour representations or entities, coloured versions of the figures are available from the Patentee upon request or from an appropriate Patent Office. A fee may be imposed if obtained from a Patent Office.

FIG. 1 provides a schematic of the production of dIgA and its secretion at mucosal surfaces as SIgA. Most dIgA produced in the submucosal tissues is subsequently bound to pIgR and transcytosed to the mucosal surface, where pIgR is cleaved to produce SIgA (or free SC), with SIgA constituting a first layer of defense against pathogens. pIgR binding is dependent on the presence of J-chain in polymeric Ig, and binding occurs to both IgM and dIgA in humans.

FIG. 2 provides a representation of the structure of dIgA1 and its interaction with pIgR which is then cleaved to give SIgA1. The associated Secretory component portion of pIgR interacts with both J-chain and the Fc domains of both individual IgA molecules.

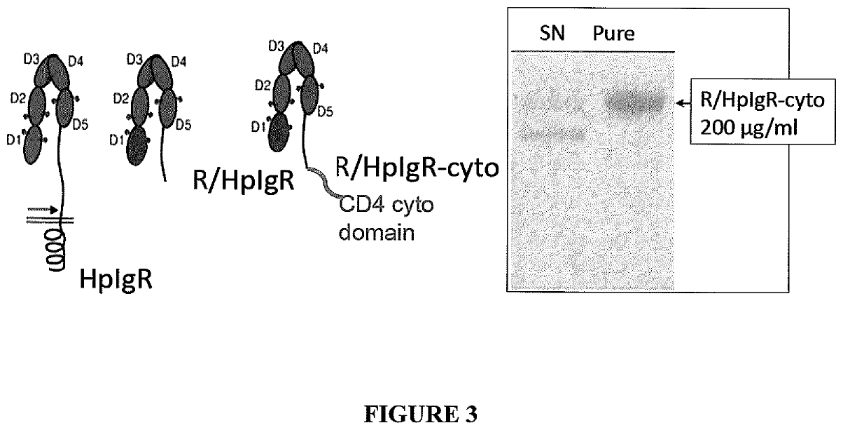

FIG. 3 provides a representation of the structure of chimeric R/HpIgR relative to human (HpIgR), and the R/HpIgR that is fused to the cytoplasmic domain of human CD4 at its C-terminus. The R/HpIgR and other forms are expressed and secreted at high levels in 293T cells, shown by the detection of R/HpIgR using coomassie brilliant blue staining of SDS-PAGE gel of the crude supernatant (SN) from transiently transfected cells. R/HpIgR-cyto is readily purified to homogeneity and high concentration by affinity chromatography with an immobilised matrix of monoclonal antibody 4B4 directed against the cytoplasmic domain of CD4 (Pure). Either pure pIgR or crude SN can be used in detection or binding of dIgA as preferred.

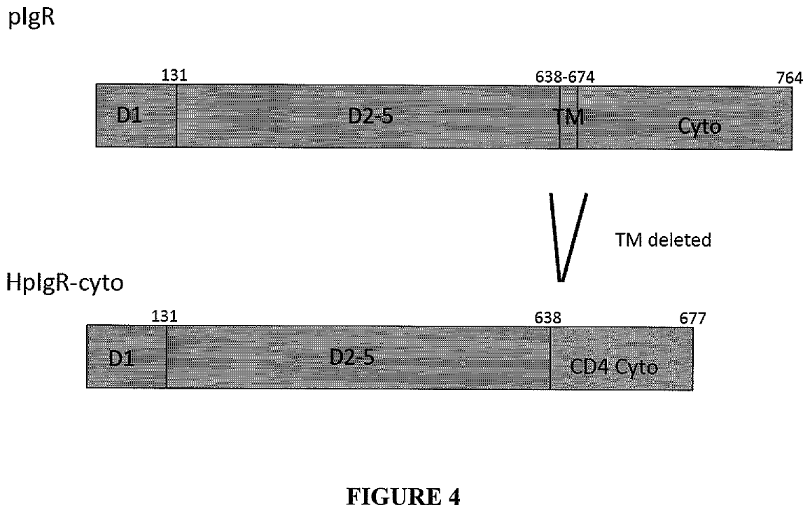

FIG. 4 provides a schematic representation of the structure of full-length (native) pIgR (TOP), relative to the recombinant form HpIgR-cyto (bottom), in which the transmembrane domain (TM) and cytoplasmic domain (cyto) of pIgR have been replaced with the cytoplasmic domain of human CD4. Because of the deletion of the TM domain, the product is secreted from cells rather than being retained at the cell surface.

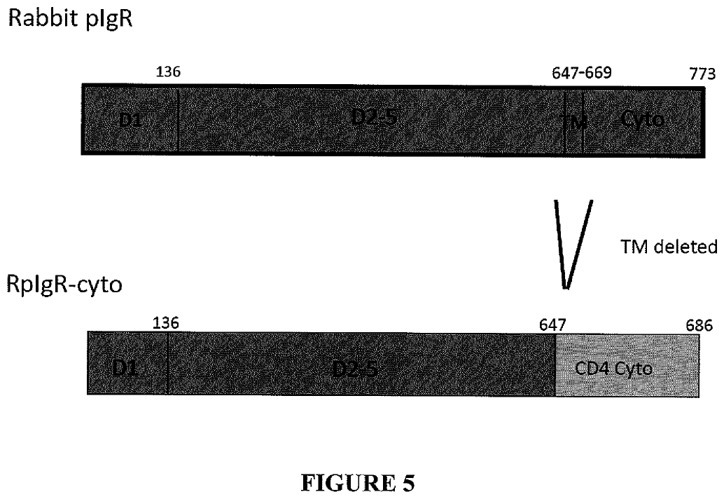

FIG. 5 provides a schematic of the structure of full-length (native) rabbit pIgR (TOP), relative to the recombinant form RpIgR-cyto (bottom), in which the transmembrane domain (TM) and cytoplasmic domain (cyto) of rabbit pIgR have been replaced with the cytoplasmic domain of human CD4. Because of the deletion of the TM domain, the product is secreted from cells rather than being retained at the cell surface.

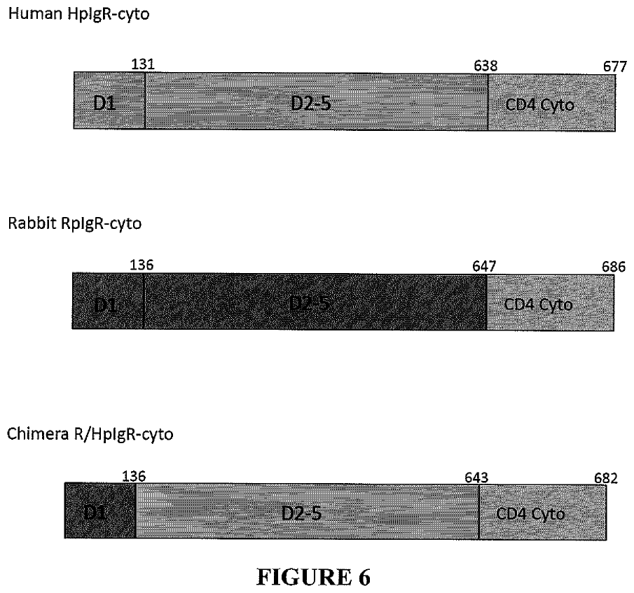

FIG. 6 provides a schematic of the structure HpIgR cyto, RpIgR-cyto, and chimeric R/HpIgR-cyto. These forms of pIgR demonstrate highly efficient binding to solid surfaces such as polystyrene ELISA plates (Nunc IMMULON® or similar) through interaction of the CD4 cyto domain with the plastic or other solid surfaces.

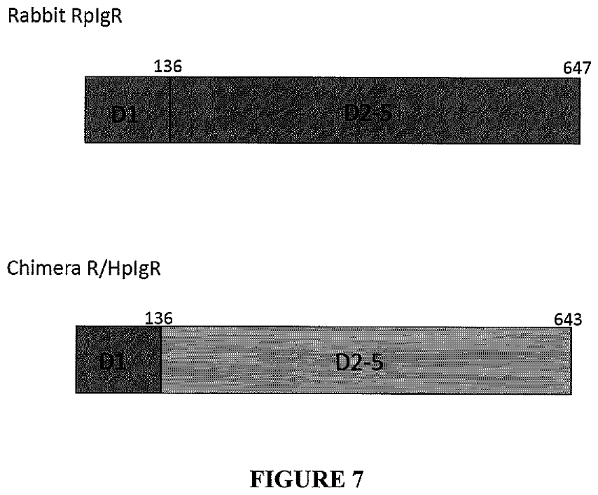

FIG. 7 provides a schematic of the structure RpIgR and chimeric R/HpIgR. In the absence of the CD4 cyto domain, the RpIgR can be detected by reactivity with antibodies against the rabbit pIgR, and the chimera R/HpIgR can be detected by reactivity with antibodies against the rabbit (domain 1) and/or human (domain 2-5) pIgR. The preferred antibodies must be able to interact with pIgR when it is bound to dIgA, not only to free pIgR,

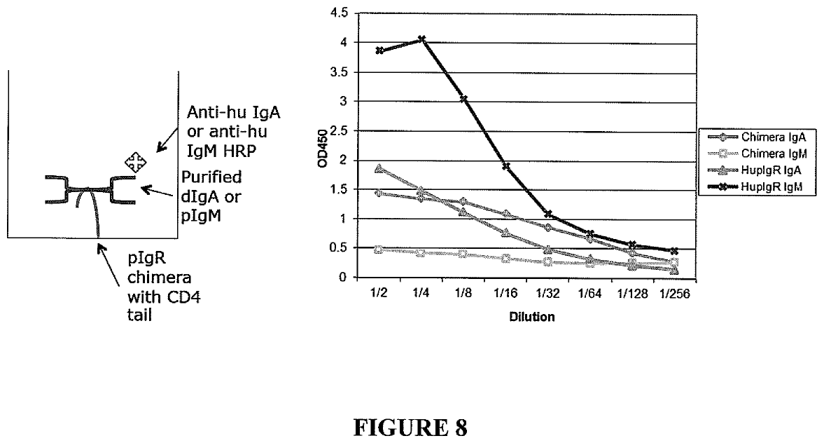

FIG. 8 provides the results of ELISA comparing the binding of HpIgR and R/HpIgR to human IgM and dIgA. HpIgR or R/HpIgR were immobilised on 96-well Nunc IMMULON® plates overnight at 4° C. Dilutions of purified human IgM or dIgA in PBS were bound to the immobilised pIgR forms overnight. After washing, the captured IgM or dIgA were detected using anti-IgM or anti-IgA conjugated to horseradish peroxidase (HRP) and colorimetric substrate TMB. The results demonstrate that HpIgR shows preferential binding to IgM (magenta) as well as binding to dIgA (green), whereas R/HpIgR shows greatly reduced binding to IgM (yellow) but retains strong binding to dIgA (blue).

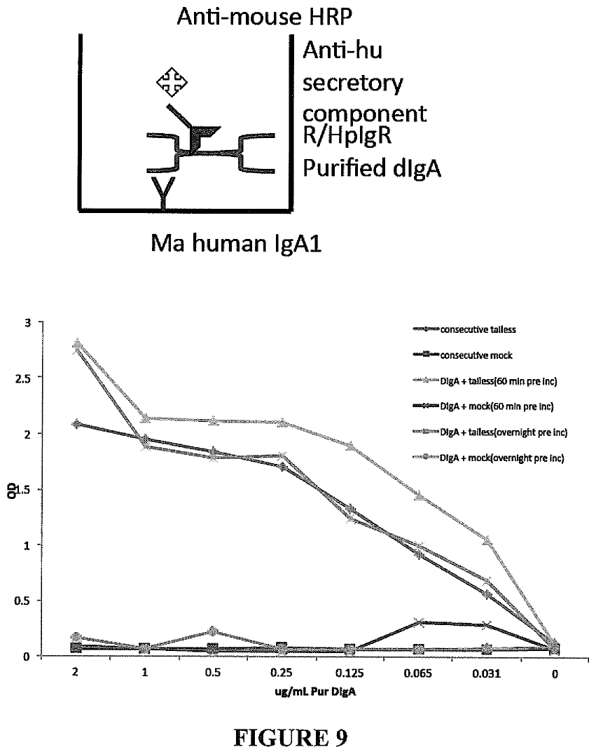

FIG. 9 provides the results of ELISA comparing the detection of immobilised dIgA using R/HpIgR. Dilutions of purified dIgA or no dIgA (mock) were immobilised on 96-well Nunc IMMULON® plates that were previously coated with anti-IgA, so that the dIgA was bound to the plate by antibody-antigen interaction rather than passive absorption. The dIgA was detected using R/HpIgR (“tailless”) or no pIgR (“mock”), anti-secretory component and anti-mouse HRP and TMB substrate. The results demonstrate R/HpIgR is able to detect dIgA at the lowest concentration tested (31 ng/ml) with strong signal in ELISA with negligible background.

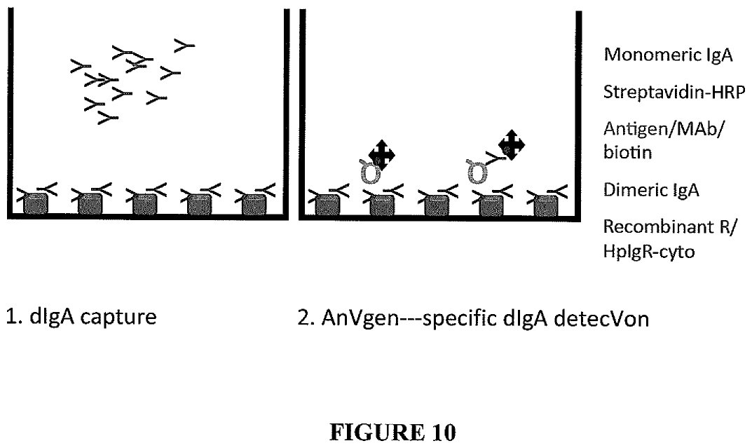

FIG. 10 is a schematic of one preferred experimental approach for detecting the presence of antigen-specific dIgA in a sample such as human serum or plasma. Recombinant R/HpIgR-cyto is immobilised on the ELISA plate, and incubated with serum or other samples. Dimeric IgA is captured on the solid phase, and after washing to remove other sample components (such as IgA and IgG that are not captured, left), the presence of antigen-specific dIgA is detected by sequential addition of antigen that is either biotinylated, or reacted with a biotinylated monoclonal antibody against the antigen, and streptavidin-HRP. In this way, any antigen that is immobilised by reaction with antigen-specific dIgA will give a signal through the biotin-streptavidin interaction.

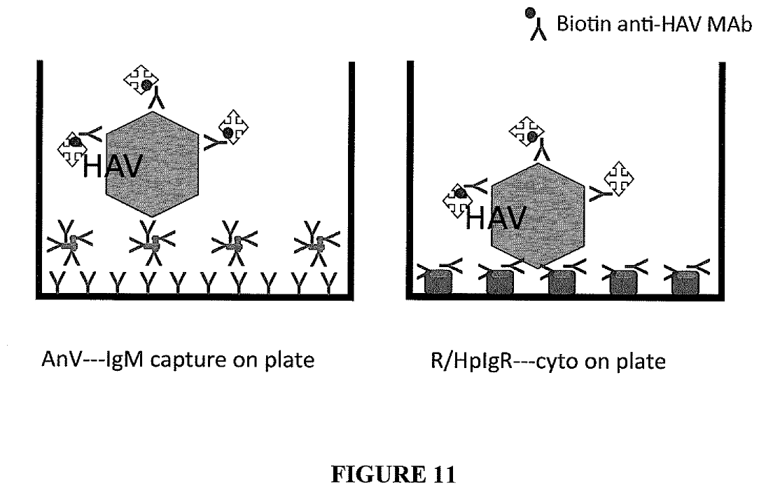

FIG. 11 is a schematic of one preferred experimental approach for detecting the presence of hepatitis A virus-specific dIgA in serum (right), compared to detection of HAV-specific IgM using the standard method of anti-IgM capture (left).

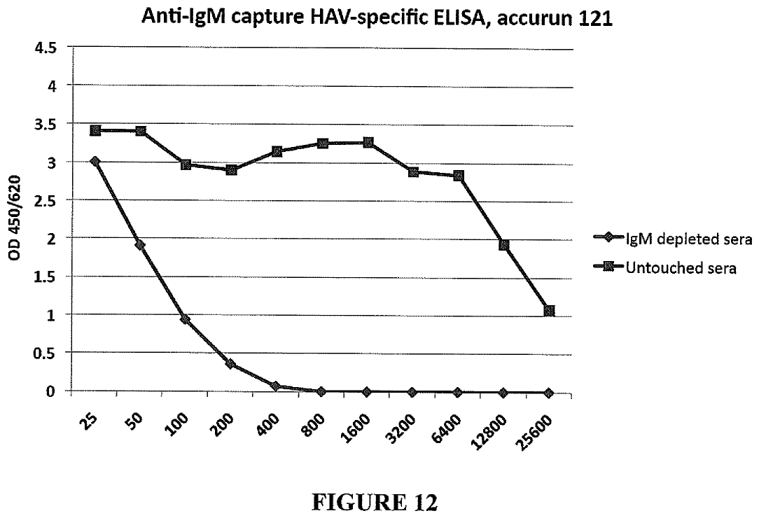

FIG. 12 provides the results of ELISA demonstrating the detection of HAV-specific IgM in IgM capture, using serial dilutions of serum from a patient with acute HAV infection (ACCURUN® HAV panel sample 121). The serum sample is either untouched before dilution (untouched, purple) or substantially depleted of IgM using Capture-Select IgM (BAC) (red). The results show that this IgM depletion method reduces the level of HAV-specific IgM in the sample by around 256-fold compared to untouched serum.

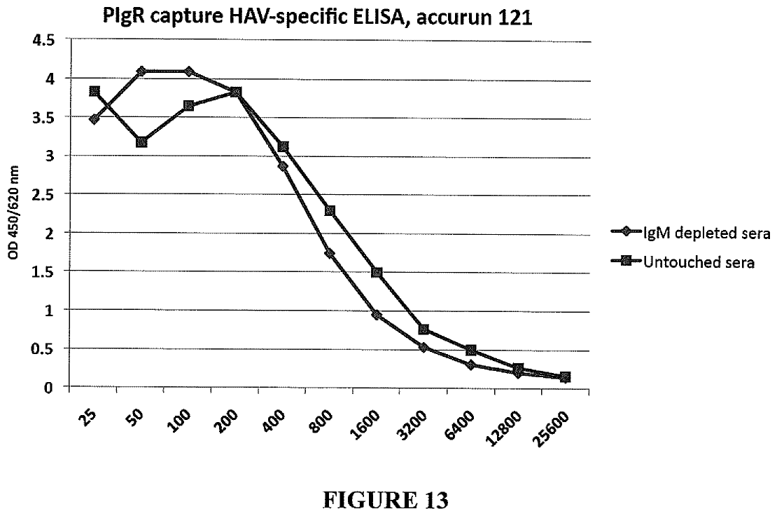

FIG. 13 provides the results of ELISA demonstrating the detection of HAV-specific dIgA in R/HpIgR capture, using serial dilutions of serum from a patient with acute HAV infection (ACCURUN® HAV panel sample 121). The serum sample is either untouched before dilution (untouched, purple) or substantially depleted of IgM using Capture-Select IgM (BAC) (red). The results show firstly the strong signal that is obtained demonstrating the detection of HAV-specific dIgA, and secondly that this signal is specific for dIgA not IgM because the IgM depletion method did not substantially reduce the level of HAV-specific reactivity compared to untouched serum, in contrast to the results shown in FIG. 12 for IgM detection.

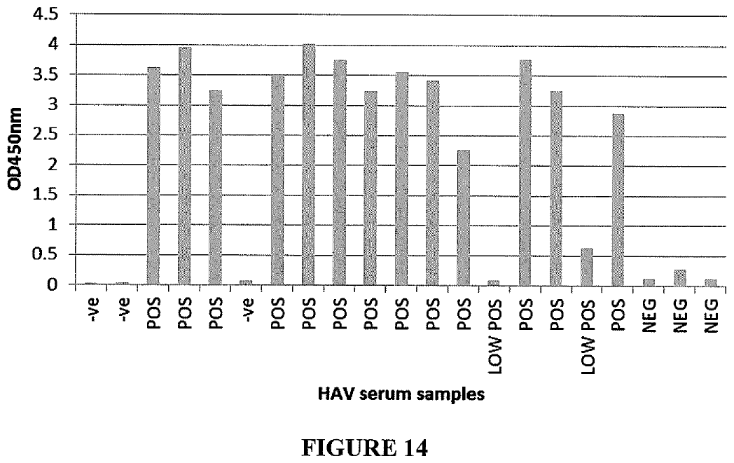

FIG. 14 provides the results of ELISA demonstrating the detection of HAV-specific dIgA in R/HpIgR capture, using sera from patients with or without acute HAV infection (ACCURUN® HAV panel, positive (POS), low positive (LOW POS), or negative (NEG)). The results show the strong detection of HAV-specific dIgA in all POS samples and in one of two LOW POS samples, with minimal background reactivity in NEG samples, demonstrating the utility of R/HpIgR capture of antigen-specific dIgA for the diagnosis of acute HAV infection.

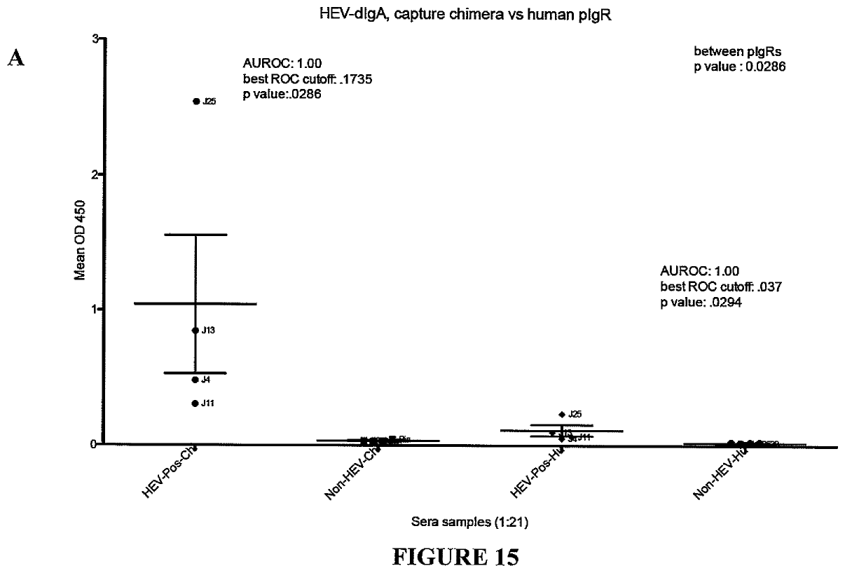

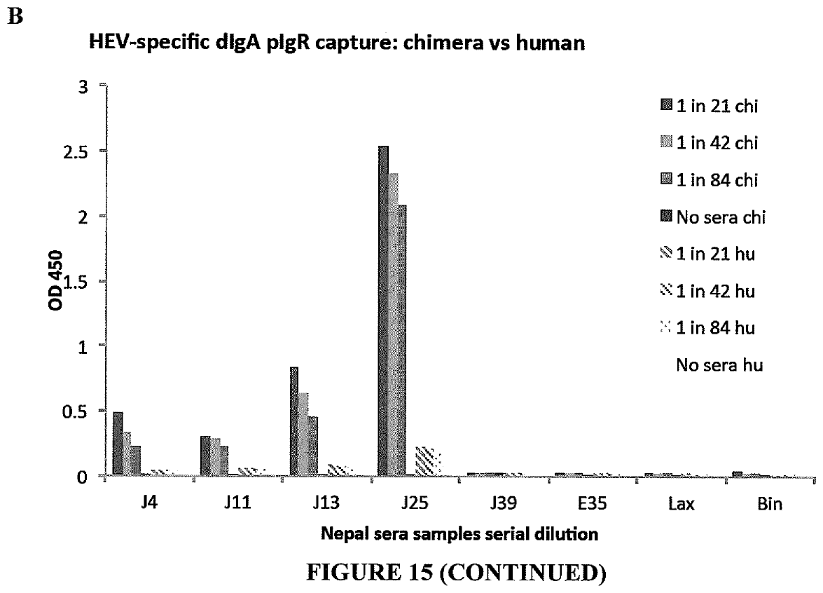

FIG. 15 provides the results of ELISAs demonstrating the detection of hepatitis E virus (HEV)-specific dIgA in R/HpIgR capture or HpIgR capture, using sera from patients with or without acute HEV infection. On the left, the ELISA OD of individual samples is shown, demonstrating the utility of R/HpIgR capture of antigen-specific dIgA for the diagnosis of acute HEV infection, with lower but still significant utility of HpIgR capture for this purpose, and negligible background reactivity in either example. On the right, the reactivity of serial dilutions of each serum sample is shown, confirming the utility of R/HpIgR capture and lower utility of HpIgR capture for diagnosis of acute HEV infection. It is likely that the lower utility of HpIgR capture in these examples is due to the much higher overall concentration of IgM in serum versus dIgA, resulting in only a low proportion of the IgM captured by HpIgR being specific for HEV.

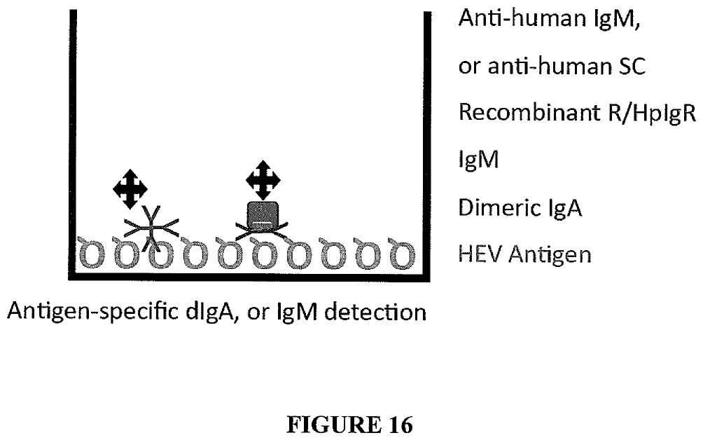

FIG. 16 is a schematic of a second preferred method for detection of antigen-specific dIgA (or IgM), in which antigen is coated directly onto the ELISA plate (in this case, hepatitis E virus (HEV) antigen). Serum samples are applied to the plate and antigen-specific antibodies, including IgM and dIgA, bind to the antigens and are then detected with either anti-IgM HRP, or R/HpIgR and anti-human SC HRP. After final washing, signal is generated with TMB substrate.

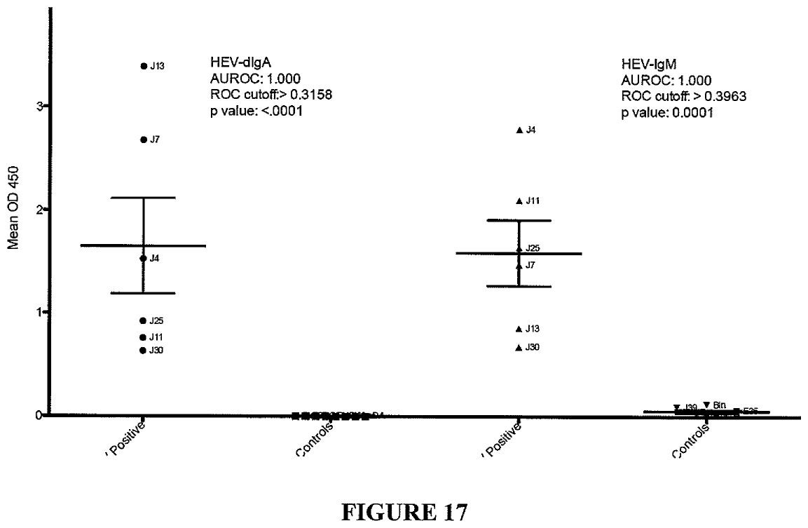

FIG. 17 provides the results of a comparison of HEV-specific dIgA versus HEV-specific IgM. Both methods are able to detect all HEV-infected patients with strong ELISA signals, compared to extremely low background for control (HEV-negative) patients in the dIgA assay, and low background in the IgM assay. Notably, some samples show higher levels of dIgA compared to IgM (sample J13, J7), while others show higher levels of IgM compared to dIgA (J4, J11). This demonstrates that the dIgA and IgM responses in patients are independent, and suggests that a combination of both IgM and dIgA detection may be useful in some desirable assay formats.

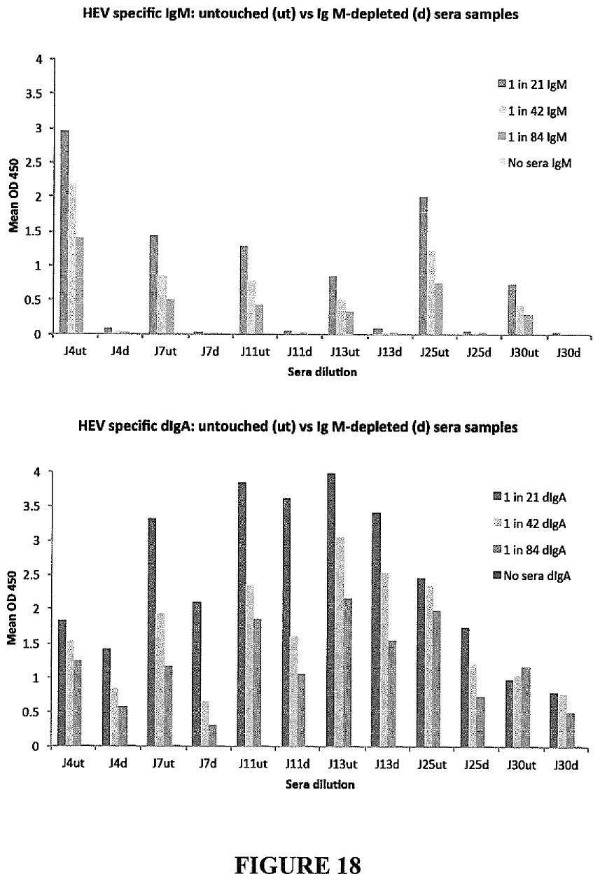

FIG. 18 provides the results of a comparison of HEV-specific dIgA versus HEV-specific IgM using sera that are either untouched, or substantially depleted of IgM using Capture-Select IgM, and then serially diluted. The results confirm that the IgM assay is specific for IgM, because the reactivity is ablated by IgM depletion, whereas the dIgA assay is predominantly specific for dIgA and not IgM, because the reactivity is only slightly affected by IgM depletion.

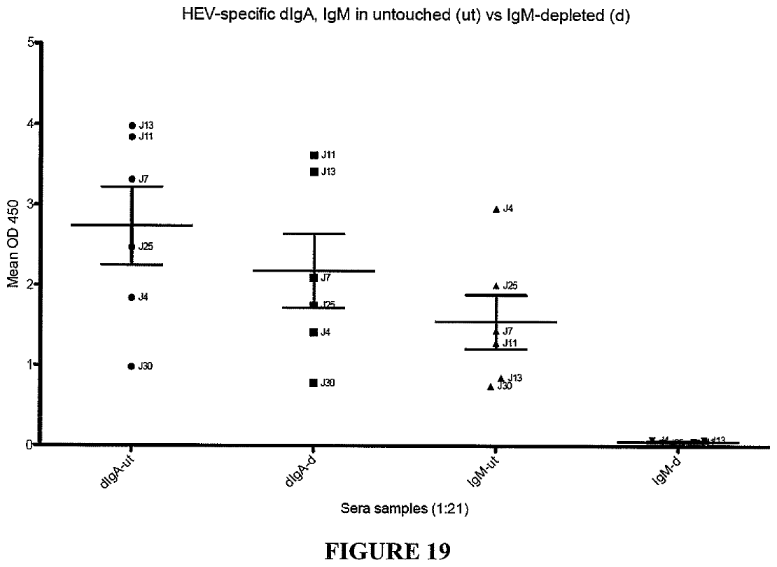

FIG. 19 provides the results of a comparison of HEV-specific dIgA versus HEV-specific IgM using sera that are either untouched, or substantially depleted of IgM using Capture-Select IgM. The results confirm that the IgM assay is specific for IgM, because the reactivity is ablated by IgM depletion, whereas the dIgA assay is predominantly specific for dIgA and not IgM, because the reactivity is only slightly affected by IgM depletion.

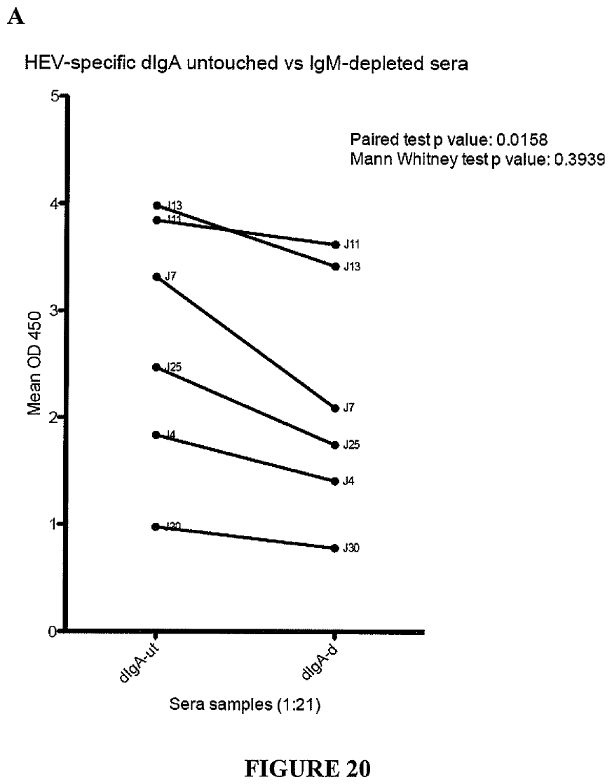

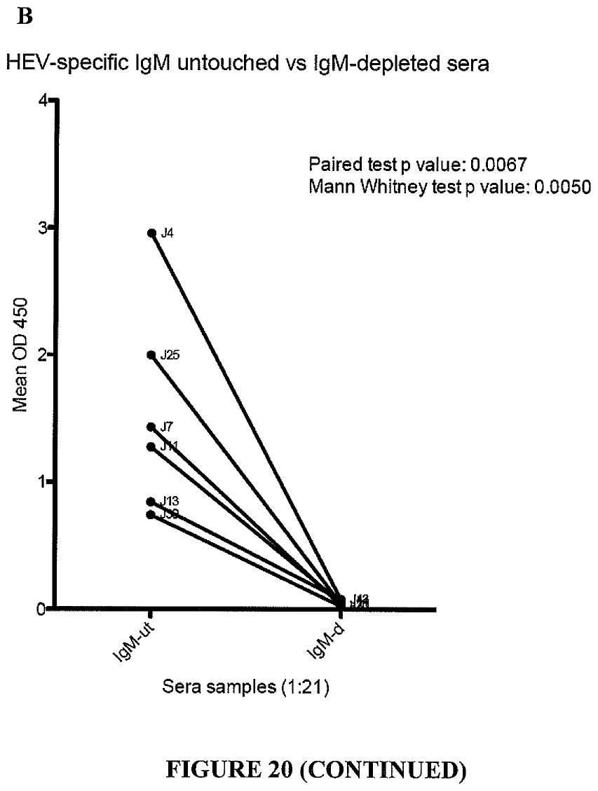

FIG. 20 provides the results of comparison of HEV-specific dIgA versus HEV-specific IgM using sera that are either untouched, or substantially depleted of IgM using Capture-Select IgM. The results confirm that the IgM assay is specific for IgM, because the reactivity is ablated by IgM depletion, whereas the dIgA assay is predominantly specific for dIgA and not IgM, because the reactivity is only slightly affected by IgM depletion. The reduction in dIgA activity following IgM depletion is statistically significant when using a paired T-test to compare samples before and after depletion, but is not significant when using a Mann-Whitney test to compare the overall sample sets before and after depletion.

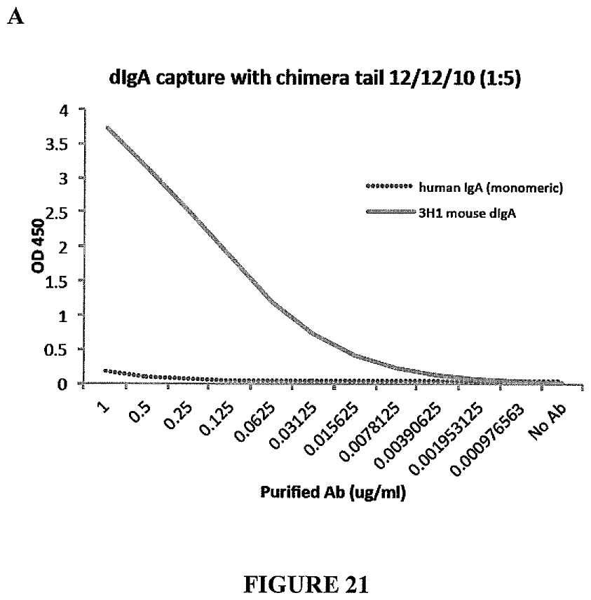

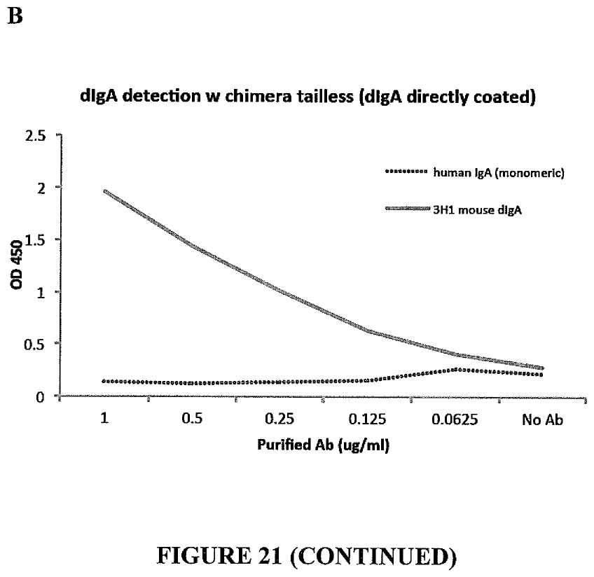

FIG. 21 provides the results of ELISA demonstrating that the R/HpIgR can be used in both capture (A) and detection (B) of mouse dimeric IgA, with negligible background reactivity to monomeric (human) IgA. A. Dilutions of purified mouse IgA monoclonal antibody 3H1 (anti-HAV) or purified monomeric human IgA were coated on plates and detected with R/HpIgR and anti-SC antibodies. B. R/HpIgR was coated on plates and dilutions of purified mouse IgA monoclonal antibody 3H1 or purified monomeric human IgA were allowed to bind overnight, then detected with anti-mouse IgA or anti-human IgA. The binding of IgA from diverse species to human or rabbit pIgR is known in the art, and this demonstrates that the novel pIgR strategy described herein has utility for diagnosis of infection in other species. It is also useful for purification of dIgA from other species—e.g., purification of mouse, rabbit or rate IgA monoclonal antibodies versus jacalin agarose.

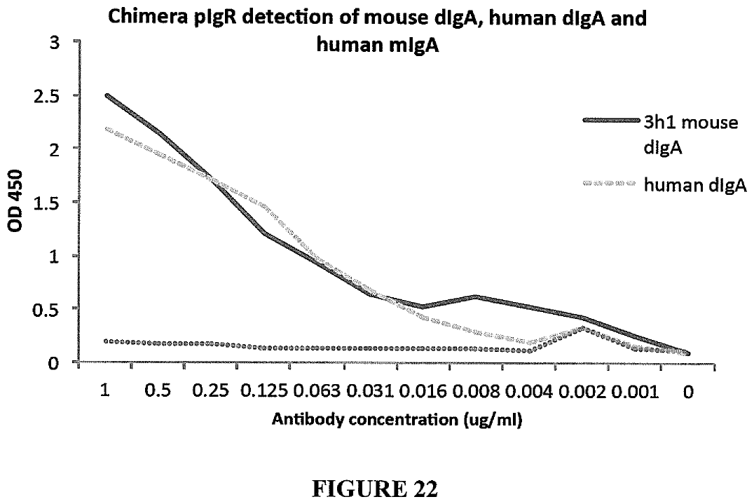

FIG. 22 provides the results of ELISA demonstrating that the R/HpIgR is equally effective for detection of mouse dimeric IgA and human dimeric IgA, with negligible background reactivity to monomeric human IgA. A. Dilutions of purified mouse IgA monoclonal antibody 3H1 (anti-HAV) or purified dimeric or monomeric human IgA were coated on plates and detected with R/HpIgR and anti-SC antibodies.

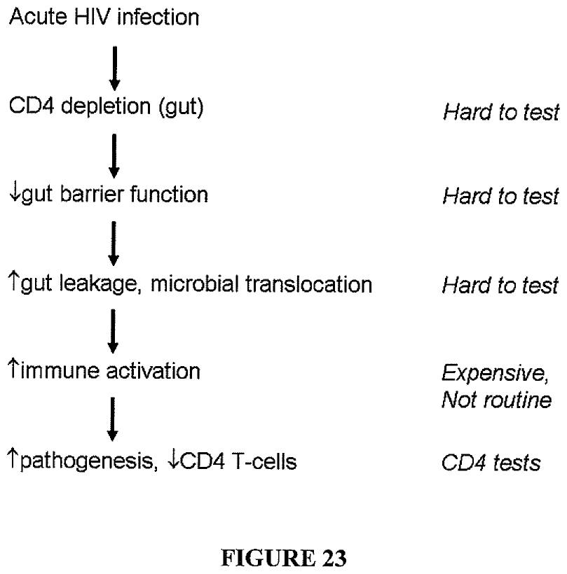

FIG. 23 provides a model outlining the pathogenic consequences of acute human immunodeficiency (HIV) infection, which leads to rapid CD4 depletion in the gut as well as the periphery, with a subsequent reduction in gut barrier function, increased leakage of gut contents and microbial translocation, leading to increased immune activation which drives pathogenesis and further reduction of CD4 T-cell levels (Brenchley et al, Nat Med 12:1365-1371, 2006). There is an unmet need for simple, standardised assays that can detect one or more of these steps in the pathogenesis pathway so that appropriate interventions can be provided to patients, with only CD4 testing having been integrated into the standard of care for HIV-infected patients. Detection of CD4 depletion in the gut requires endoscopy; detection of decreased gut barrier function requires complicated sugar challenge studies or other methods; detection of gut leakage and microbial translocation can be achieved using markers such as bacterial LPS or 16sRNA in serum but results are highly variable due in part to the wide variation in gut microbiota between individuals; immune activation requires complex Flow cytometry protocols that are difficult to standardise across instruments/operators.

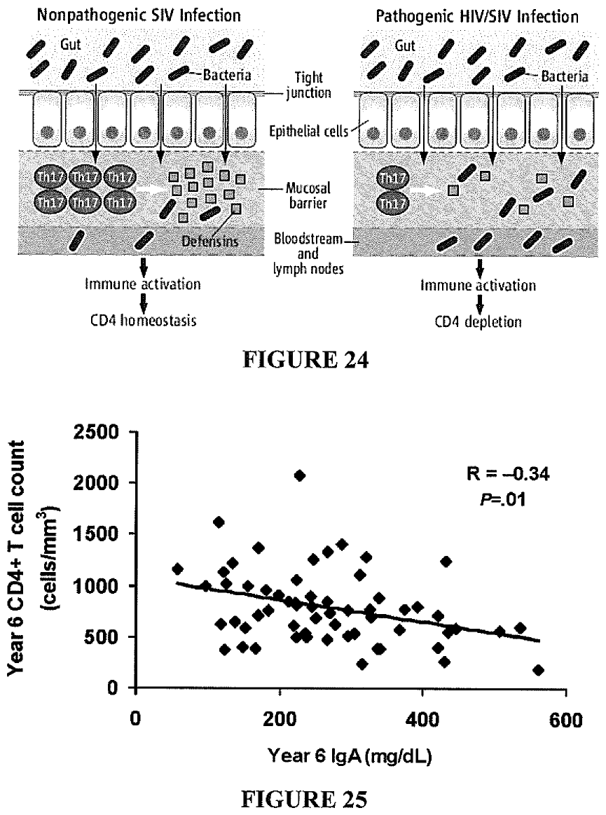

FIG. 24 is a schematic of the increase in microbial translocation due to gut leakage induced by pathogenic HIV or SIV infection, compared to normal low levels of translocation in nonpathogenic SIV infection,

FIG. 25 illustrates one expected consequence of increased microbial translocation is the induction of increased IgA responses due to mucosal antigen exposure. French et al., J Infect Dis. 200(8): 1212-1215, 2009 demonstrated that indeed the total level of IgA in HIV patients after 6 years of follow up was inversely correlated with the level of CD4 T-cells in patients undergoing highly active antiretroviral therapy, suggesting that even in patients being treated with the most effective current antiviral therapies, microbial translocation contributes to pathogenesis. However these results also show that total IgA is highly variable between individuals, and does not provide a prognostic marker that can be used in management of individual patients.

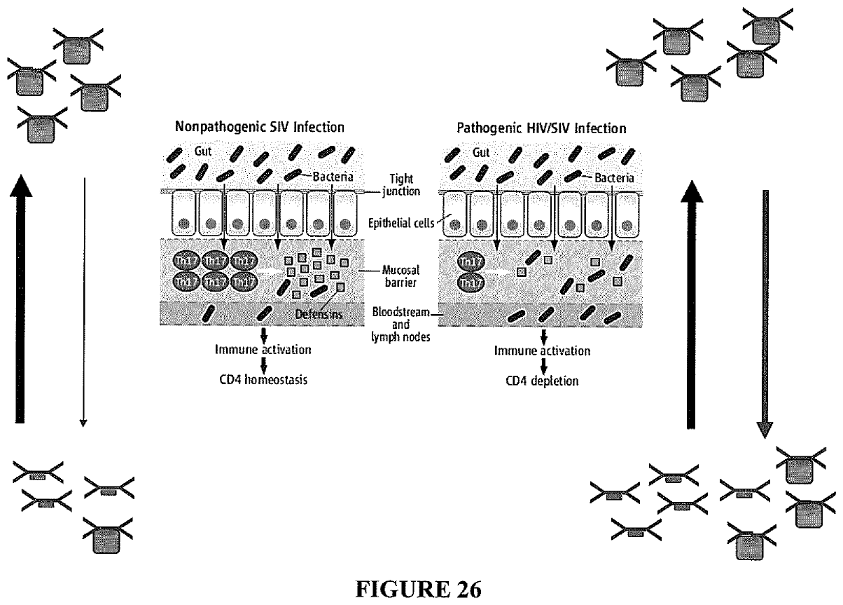

FIG. 26 provides a schematic illustration of the increase in microbial translocation due to gut leakage induced by pathogenic HIV or SIV infection, compared to normal low levels of translocation in nonpathogenic SIV infection, showing the expected effect on dimeric IgA and secretory IgA levels in the plasma compartment. Under normal conditions or nonpathogenic SIV infection, gut barrier integrity is maintained and the level of SIgA in the lumen of the gut reflects the amount of its precursor dIgA in the lamina propria. Only a minimal amount of SIgA is returned to the plasma compartment, either through active transport by M-cells in the gut, or a small amount of gut leakage. The amount of leakage or active transport can be estimated by comparing the serum/plasma concentration of SIgA to that of its precursor dIgA, giving a ratio of SIgA/dIgA. Under conditions of pathogenic HIV or SIV infection, or other physiological challenges that result in gut leakage, the total amount of dIgA is likely to be somewhat elevated and may lead to higher levels of SIgA secretion into the lumen. However a much higher proportion of SIgA will be returned to the plasma compartment due to passive leakage through the compromised gut barrier, resulting in an elevated SIgA/dIgA ratio.

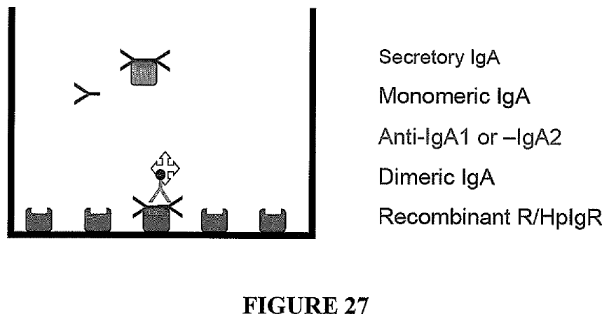

FIG. 27 provides a schematic of one of several typical assays that can be used to measure the relative amount of different IgA forms in order to estimate the SIgA/dIgA ratio. In this example, the amount of dIgA is measured by capture of dIgA using R/HpIgR, and detection using monoclonal antibodies against either IgA1, or IgA2, or against both IgA subclasses. Monomeric IgA does not bind to pIgR; SIgA does bind to R/HpIgR but with lower affinity than dIgA and can be removed by washing with 3.5 M urea if desired. SIgA is measured in the same way but using anti-SC antibody capture instead of R/HpIgR. The SIgA/dIgA ratio is then calculated as a simple ratio of the assay reactivities for SIgA and dIgA.

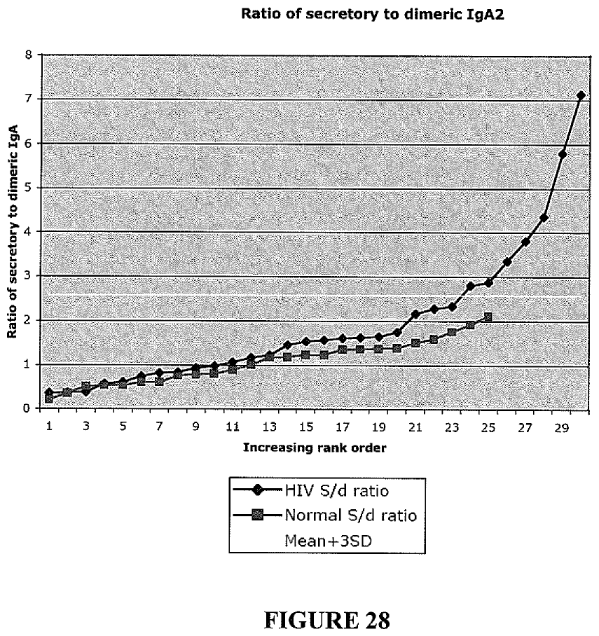

FIG. 28 provides the results of ELISA demonstrating the detection of highly elevated SIgA2/dIgA2 (S/d) ratios in a proportion of HIV-infected patients, compared to the majority of HIV-infected patients and all control subjects (magenta). The assay cutoff for elevated SIgA/dIgA was set as the mean plus 3 standard deviations of the SIgA/dIgA ratio among non-HIV control subjects, and 7/30 HIV-infected subjects showed SIgA/dIgA ratios above this cutoff. Notably, the range of SIgA/dIgA ratios among normal subjects is smaller than the range for SIgA or dIgA alone, because the role of dIgA as the precursor of SIgA provides a normalising effect for each patient.

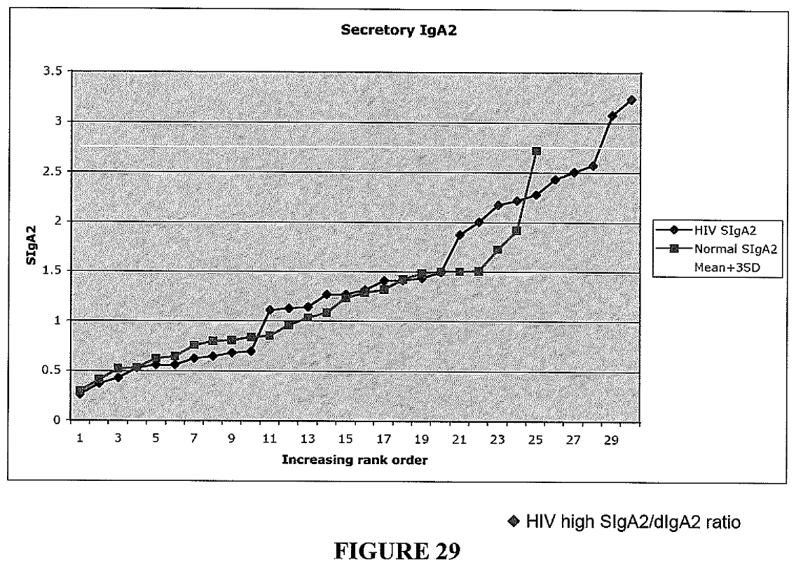

FIG. 29 provides the results of ELISA demonstrating the total amount of SIgA in patient and control sera (arbitrary units). The amount of SIgA2 in normal patients varies over an 11-fold range, but all normal controls fall within a cutoff of the mean plus 3 standard deviations. The amount of SIgA2 in HIV-infected patients varies over a slightly larger range (16-fold), but only 2/30 patients are above the cutoff range. Among the HIV-infected patients, those patients who demonstrated elevated SIgA2/dIgA2 ratios in FIG. 28 are indicated with red markers. It can be seen that these patients with elevated SIgA2/dIgA2 ratios are found throughout much of the normal range of the total SIgA2 signal, and cannot be distinguished from the normal controls on the basis of the total SIgA2 alone. This confirms the utility of using SIgA/dIgA ratios because the role of dIgA as the precursor of SIgA provides a normalising effect for each patient. The R/HpIgR system provides the utility for measuring this ratio.

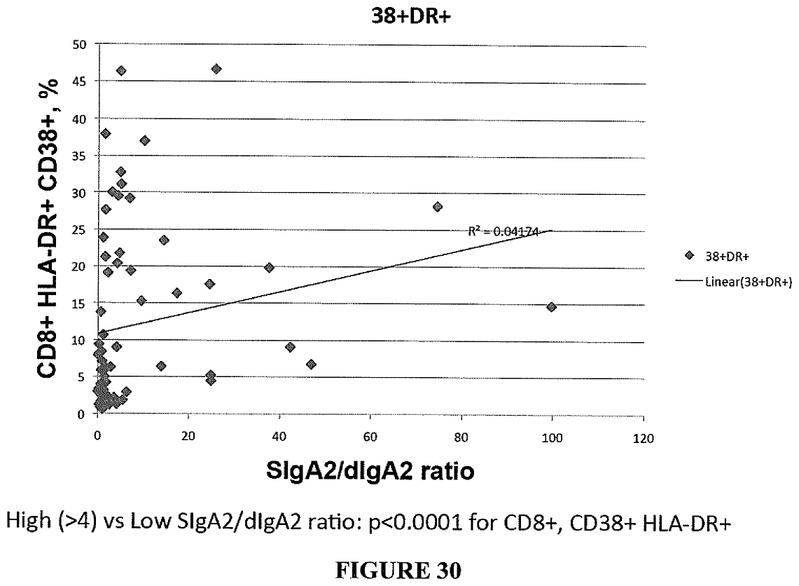

FIG. 30 illustrates the correlation of SIgA2/dIgA2 ratio versus the immune activation marker, CD8+HLA−DR+CD38+ T-cells, in a different HIV-infected population to that shown in FIGS. 28 and 29. While the overall correlation is low, it is apparent that patients with SIgA2/dIgA2 ratios of >4 in this experiment have elevated levels of immune activation markers (p<0.0001).

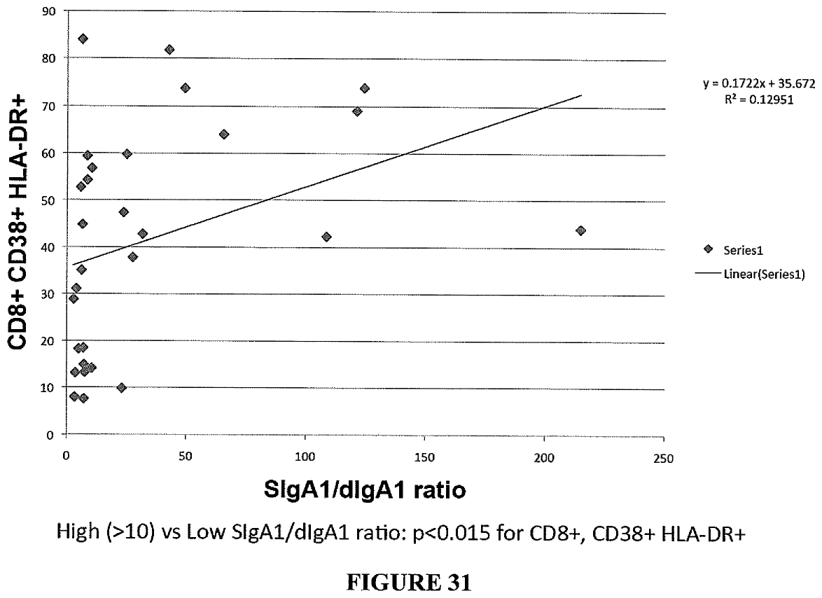

FIG. 31 illustrates the correlation of SIgA1/dIgA1 ratio versus the immune activation marker, CD8+HLA−DR+CD38+ T-cells, in the same population as FIG. 30. While the overall correlation is lower again than for IgA2, it is apparent that patients with SIgA1/dIgA1 ratios of >10 in this experiment have elevated levels of immune activation markers (p<0.015). The lower correlation for IgA 1 and higher cutoff ratio (10 versus 4) for significance highlights the value of specifically measuring IgA2 because of its predominant site of synthesis in the gut, being the tissue in which leakage of SIgA is likely to be clinically relevant marker of gut leakage and immune activation.

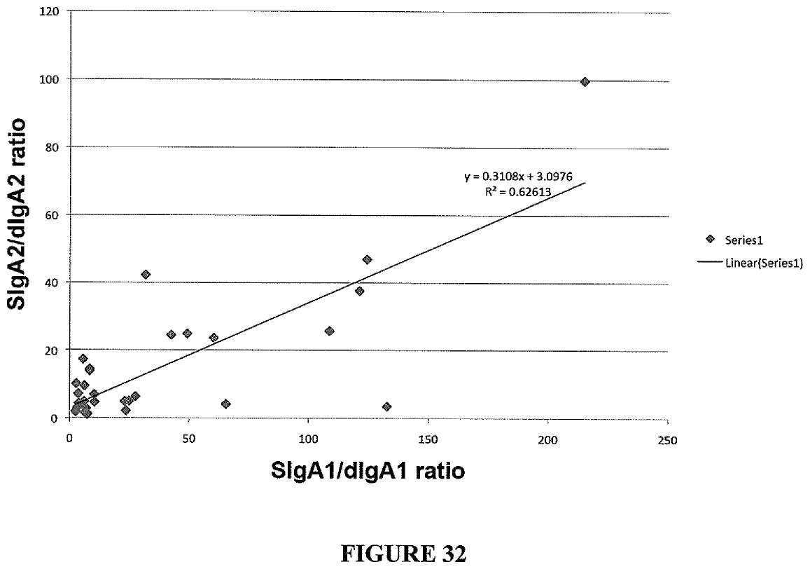

FIG. 32 illustrates the correlation of SIgA1/dIgA1 ratio versus SIgA2/dIgA2 ratio, in the same population as FIGS. 30 and 31. While SIgA1/dIgA1 ratios are significantly correlated with SIgA2/dIgA2, it is notable that there are some patients with highly elevated SIgA1/dIgA1 ratios and relatively low SIgA2/dIgA2 ratios. This suggests that there may be some value in measuring IgA1, or total IgA, in addition to IgA2 in the calculation of SIgA/dIgA ratios as a measure of gut leakage and immune activation.

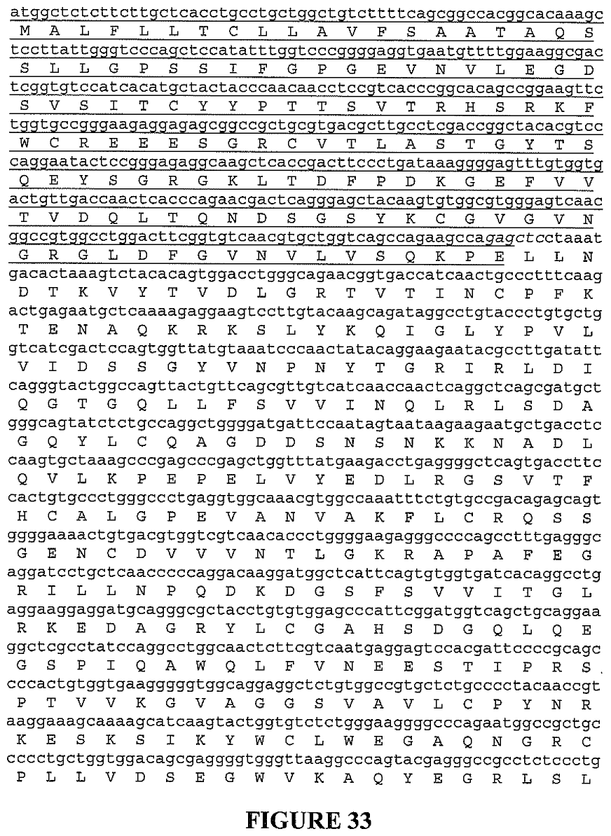

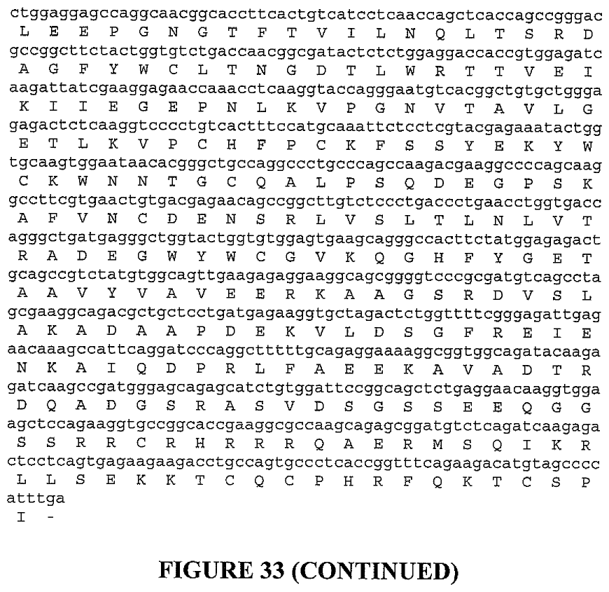

FIG. 33 provides the nucleotide sequence (SEQ ID NO: 15) and amino acid sequence (SEQ ID NO: 16) of CHIMERA-CD4 cyto (R/HpIgR-cyto) showing rabbit sequences underlined and human sequences in black, and CD4 cyto sequences in grey.

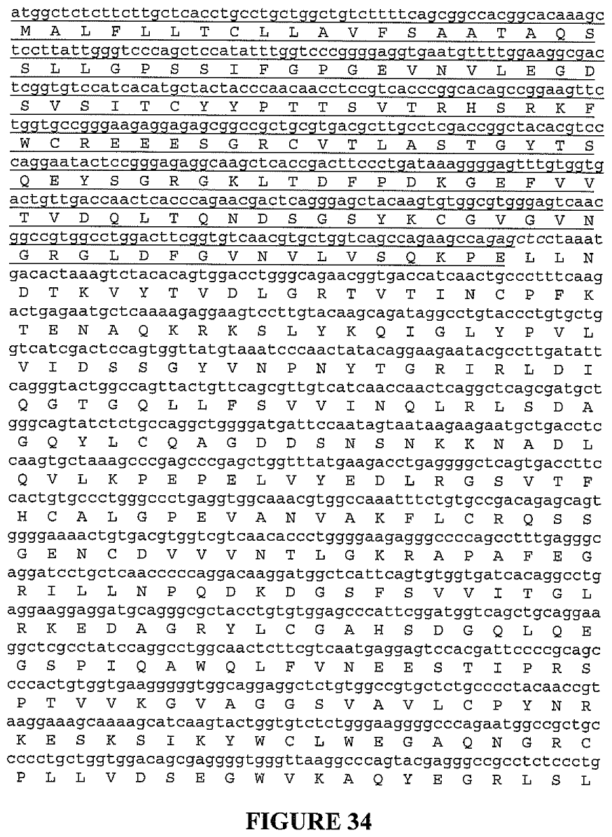

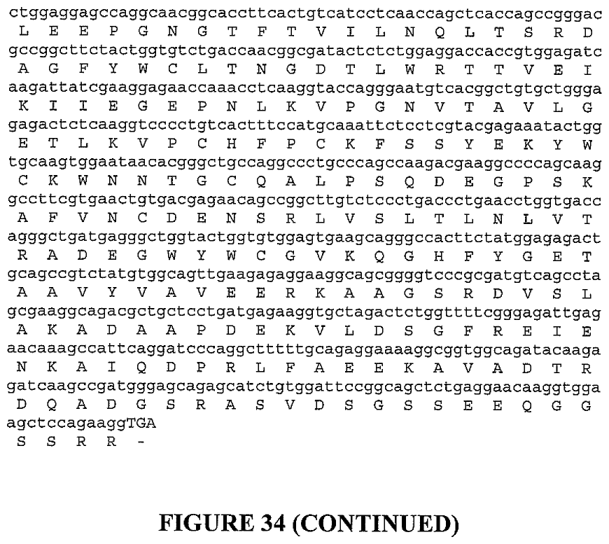

FIG. 34 provides the nucleotide sequence (SEQ ID NO: 5) and amino acid sequence (SEQ ID NO: 6) of CHIMERA (R/HpIgR) showing rabbit sequence underlined and human sequence in black.

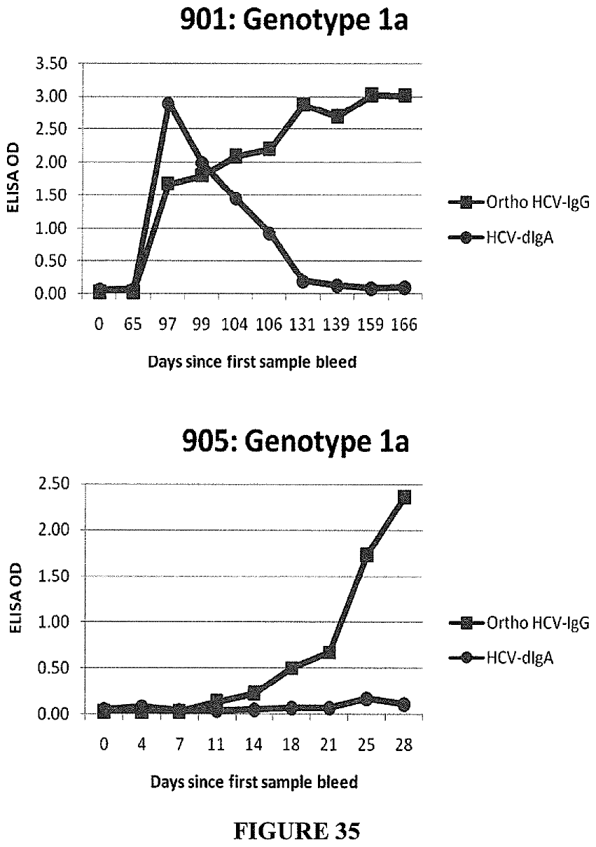

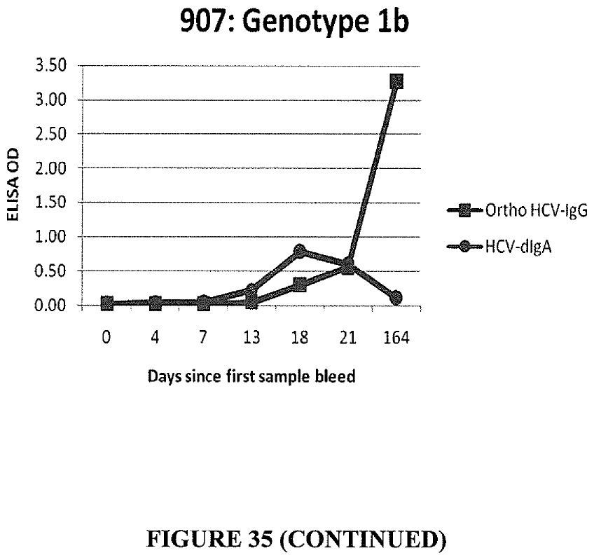

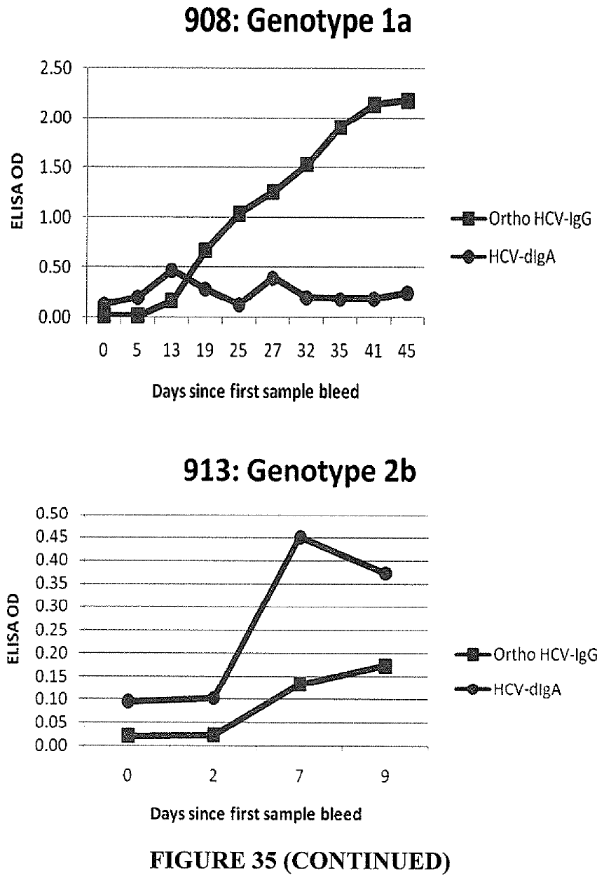

FIG. 35 provides graphical and tabulated date showing hepatitis C virus specific dIgA detection. In addition to the acute, self-limiting hepatitis A virus and hepatitis E virus which are transmitted from person to person via fecal-oral routes, there are at least three other viruses that cause viral hepatitis which is commonly chronic and leading to severe long-term disease, and where the viruses are considered to be blood-borne, namely hepatitis B, hepatitis C and hepatitis D. In this example, it can be seen that even for a chronic infection such as hepatitis C virus, HCV-specific dIgA is detectable for only a relatively short time after infection, up to around 100 days after the last blood sample that tested negative for HCV, suggesting that HCV-specific dIgA may be a marker of acute infection. While only 4 out of 5 (80%) of HCV patients had detectable HCV-specific dIgA in this assay, it will be evident to those skilled in the art that there are a variety of methods that could be used to increase the sensitivity of this assay for detection of acute-phase HCV, which will be useful in determining the best options for treating patients with antiviral drugs or interferon-containing medications. It will also be evident that since virus-specific dIgA is found in the diverse range of diseases including hepatitis A, hepatitis E, hepatitis C and tuberculosis, it is reasonable to assume that it may be detectable, and of utility in diagnostic and therapeutic protocols, for any other infectious disease.

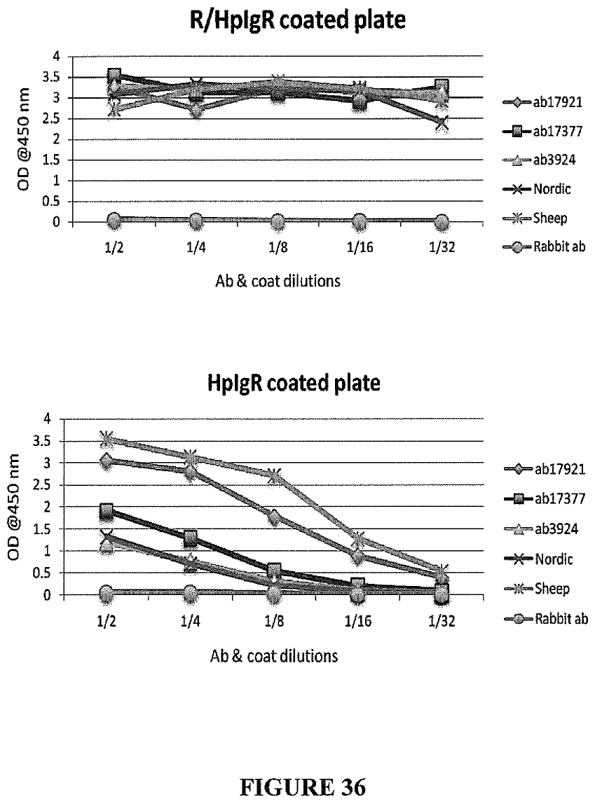

FIG. 36 provides a graphical representation of data comparing different commercially available antibodies for binding to RiElpIgR or HpIgR. It will be apparent to those skilled in the art that there are many different monoclonal and polyclonal antibodies directed against the pIgR (also known as secretory component or SC) that may be used for the detection of bound pIgR, and thus antigen-specific or total dIgA or IgM, in a variety of assay methods. In this example, it can be seen that several commercially available antibodies can be used for this purpose, whereby serial dilutions of either recombinant expressed chimeric R/HpIgR or recombinant expressed human HpIgR was coated on polystyrene ELISA plates, and detected using serial dilutions of mouse monoclonal or sheep polyclonal antibodies, and detected with appropriate anti-species antibodies. Note that the R/HpIgR is present at higher concentrations, and so there is no titration of the assay signal in this experiment, whereas with the HpIgR which is present at lower concentrations, the titration of antibody reactivity reveals that commercial Abcam monoclonal antibody 17921 is approximately equivalent to sheep polyclonal antibody, while Abcam 17377 and 3924 appear to be less reactive, as does the Nordic Immunology monoclonal against SC. The rabbit antibody is a negative control. These and other antibodies can be selected according to their binding to either free pIgR forms, or pIgR forms already bound to dIgA or pIgM, to fit the purpose needed in detection of R/HpIgR or HpIgR or dIgA-binding variants thereof accordingly.

### DESCRIPTION OF PARTICULAR EMBODIMENTS

Throughout this specification and the claims which follow, unless the context requires otherwise, the word “comprise”, and variations such as “comprises” and “comprising”, will be understood to imply the inclusion of a stated integer or element or method step or group of integers or elements or method steps but not the exclusion of any other integer or element or method step or group of integers or elements or method steps.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise.

Nucleotide and amino acid sequences are referred to by a sequence identifier number (SEQ ID NO:). The SEQ ID NOs: correspond numerically to the sequence identifiers <400>1 (SEQ ID NO:1), <400>2 (SEQ ID NO:2), etc. A summary of sequence identifiers is provided in Table 1. A sequence listing is provided at the end of the specification.

As described herein, the present disclosure provides an antibody capture process comprising determining the level or presence of dimeric or polymeric IgA (dIgA) in a biological sample. The process of the present invention may be practised by detecting only the level or presence of dIgA, or it may be practised in combination with protocols to determine the level or presence of one or more further antibody forms (e.g., monomeric, dimeric, polymeric or pentameric complexes), classes (isotypes) or subclasses (e.g., dIgA1, dIgA2, SIgA2, etc.). In some embodiments, the process is practised using specific antigens that bind to dIgA molecules of interest. In other embodiments, the process may be practised to identify antigens of interest which engender dIgA responses in a subject that may be detected in a sample from a subject. In some embodiments, the process enables the development inter alia of diagnostic assays and therapeutic protocols that are useful for assessing secretory IgA responses at mucosal surfaces and associated tissues such as gut-associated lymphoid tissue (GALT).

The present process is predicated in part on the ability to detect dIgA or dIgA and IgM with high sensitivity and specificity in binding assays using a recombinant polymeric Ig receptor (pIgR). The present process employs recombinant pIgR or recombinant variants of pIgR that bind dIgA and IgM, as well as recombinant pIgR or variants of pIgR that preferentially bind dIgA and substantially fail to bind IgM.

Accordingly, the present specification provides an antibody-capture process comprising detecting or capturing a precursor to secretory dIgA (SIgA), namely dIgA. In some embodiments, the process comprises step (i) contacting a biological sample from a subject with recombinant polymeric immunoglobulin (Ig) receptor (pIgR) or an dIgA-binding variant thereof, wherein the pIgR or variant binds dIgA and IgM; or wherein the pIgR or variant binds dIgA and substantially fails to bind IgM, and wherein the pIgR substantially does not bind monomeric IgA, and step (ii) determining the level or presence of dIgA that has bound to pIgR. In some embodiments, step (ii) comprises detecting a complex between dIgA and pIgR or a complex between bound dIgA and an antigen.

Reference herein to “biological sample” includes a sample obtained from a subject comprising antibodies. The term also includes sample comprising cells expressing dIgA e.g., hybridoma cells, and samples comprising recombinant dIgA expressed from cell lines cultured in vitro. Biological samples from subjects include blood and serum samples, other bodily liquids, biopsy etc. Blood and serum samples are preferred. Reference to an “antigen” includes a protein or infection agent or part of a protein or part of an infection agent, as known in the art. Reference to RIHpIgR includes chimeric forms comprising a immunoglobulin domain from a rabbit pIgA sequence or similar dIgA-binding variant sequences derived from rat or mouse or functional (dIgA-binding) variants thereof. Thus R includes rabbit, or mouse, or rat-derived sequences.

Determining the presence or level of dIgA or pIgR or a complex between dIgA and recombinant pIgR or a complex between recombinant dIgA and an antigen may be by any convenient protocol.

A diverse range of assays are used in research, analysis, development and clinically to detect analytes of interest. Immunoassays are a particularly useful form of assay that exploits the specificity, strength and diversity of antibody-antigen type or protein-protein reactions to analyse samples and detect specific components therein. A wide range of immunoassay techniques are available, such as those described in Wild D. “The Immunoassay Handbook” Nature Publishing Group, 2001.

Methods of detecting antibody complexes, antigens or antibody-ligand complexes are well known in the art. For example, the enzyme-linked immunosorbent assay (ELISA) and radioimmunoassay (RIA) are routinely used in laboratories. These methods generally require some level of skill in laboratory techniques. A variety of methods have also been developed which require little skill and are rapid to perform, and which are therefore suitable for the detection of antibody to specific antigens at the point of care or analysis. In particular, immunochromatographic or dipstick enzyme-linked immunosorbent kits have been developed to assay for a number of infections agents.

Immunochromatographic devices are expressly contemplated comprising dIgA-binding reagents such as recombinant pIgR and further comprising antigens of interest identified as described herein as binding dIgA from infected subjects or subjects exhibiting mucosal immune activation.

Kits or immunochromatographic devices comprise, for example, reverse-flow or lateral-flow formats.

In an illustrative embodiment, a kit for assessing immune status in a biological sample from a subject is provided which employs one or more antigens of interest recognised by dIgA from subjects with active infections or conditions associated with mucosal immune activation, and employs a pIgR molecule or dIgA-binding variant thereof as a dIgA-binding reagent. In a preferred embodiment, the antigen is not a TB antigen.

In some embodiments, the kit comprises:
a) an immunographic device comprising a porous membrane operably connected to a sample portion, a test portion, and optionally a control portion; and farther comprising a sucker portion, portion comprising a pIgR molecule or dIgA-binding variant thereof, a portion comprising an antigen or agent of interest and optionally a conjugate portion; and
b) instructions for using the immunographic device to detect the presence of antigen specific dIgA antibody in the sample.

In one embodiment, the pIgR or dIgA-binding variant thereof is HpIgR or R/HpIgR or a dIgA binding variant thereof.

The subject assays may employ a wide range of suitable detection markers known in the art. In some embodiments, the detection marker may be detected using detectable characteristics of the detection marker and a wide range of detection protocols using detectable markers are well known to those of ordinary skill in the art. In some embodiments, the detection marker is directly or indirectly bound or otherwise associated with an antigen or infectious agent of interest. In other embodiments, the dIgA binding agent, such as pIgA comprises or is designed to interact with a detection marker. In some embodiments, the detection marker is connected the antigen or dIgA binding agent using binding partners known in the art such as without limitation biotin:avidin or anti-biotin antibody:biotin.

Polymeric immunoglobulin receptor (pIgR) is encoded by the PIGR gene and is expressed in mucosal epithelial cells where it facilitates uptake of dIgA and secretion of SIgA. pIgR has five immunoglobulin-like domains which bind to dIgA including to the J-chain thereof. pIgR also binds to pentameric IgM.

As determined herein it is possible to detect both dIgA and IgM with high sensitivity and specificity using recombinant human polymeric Ig receptor and parts and variants thereof (see FIG. 3). In one particular non-limiting embodiment it is shown herein that dIgA can be selectively detected using a recombinant form of the polymeric Ig receptor having at least domain 1 derived from the rabbit pIgR, for example, a chimera of rabbit (domain 1) and human (domain 2-5) pIgRs, or with all domains from rabbit pIgR. In some embodiments, the recombinant pIgR described herein are designed to bind preferentially to dIgA (plus or minus IgM), and can be used either to capture dIgA (IgM) specifically to a solid phase for reaction with an antigen of interest, in which case the pIgR does not need to have an associated detection reagent, or alternatively to detect the presence of dIgA (±IgM) bound to an antigen of interest immobilized on a solid phase, in which case the pIgR may be conveniently detected using antibodies or other reagents directed against the pIgR itself, or against epitope tags or other sites introduced into the recombinant pIgR using methods well known in the art. A further advantage of pIgR is that it shows very low background reactivity in assays, unlike typical antibody-based detection reagents.

Roe et al., J Immunol 162: 6046-52, 1999 describe a chimeric pIgR comprising immunoglobulin-like domain 1 (D1) derived from rabbit, and D2-5 derived from human pIgR which has preferential binding to dIgA over IgM. However, they do not disclose or suggest the use of this form or any other pIgR variant for detection or binding only of dIgA for diagnostic purposes or the advantages of the recombinant pIgR or dIgA binding variants disclosed herein. The substitution of human for rabbit (or mouse or rat) D1 provides preferential binding of dIgA, but it would be expected that substitution of any one or more of D2-D5 may also be substituted with the rabbit sequence to give a molecule that preferentially binds to dIgA and these variants are also encompassed. Accordingly, in some embodiments, any one or more of D1, D2, D3, D4 or D5 is substituted with rabbit, mouse or rat homologs.

In some embodiments, the recombinant pIgR lacks a transmembrane domain (ATM). In other embodiments, the recombinant pIgR lacks a cytoplasmic domain. In some embodiments, the recombinant pIgR lacks a TM domain and a cytoplasmic domain (ΔCYT). In some embodiments, recombinant pIgR comprises a substitution in the cytoplasmic domain and provides a CD4 cytoplasmic domain. Various forms of recombinant pIgR are contemplated and illustrative examples are illustrated in FIGS. 4 to 7, further described in the figure legends. The ability to design and test recombinant pIgR having a desired level of specific dIgA is illustrated in FIG. 8 and described in the legend to FIG. 8.

Phillips-Quagliata et al., J Immunol 165: 2544-2555, 2000 indicate that both rat and mouse bind predominantly dIgA, but that for mouse there is a form expressed on B-cells that has only a single amino acid change but binds both IgM and dIgA—to quote from page 2552:

“Although human pIgR and the T560 mouse pIgR bind both pIgA and IgM, rabbit (44) and rat (47) hepatocyte pIgR bind only pIgA well and do not translocate IgM into bile. Because mouse liver similarly translocates pIgA but not IgM into bile (48, 49), it is generally assumed that mouse hepatocyte pIgR resembles rat and rabbit pIgR and binds IgM poorly or not at all. If this is true, then the difference between the mouse hepatocyte and the T560 pIgR that makes the latter behave more like human pIgR must be explained. Given that the amino acid sequences of the mouse hepatocyte and T560 B cell pIgRs are the same except for the Val to Ala change in domain 2, the difference most likely reflects differential folding or glycosylation of the pIgR, probably the latter. It is easy to imagine that a bulky carbohydrate on hepatocyte-derived pIgR could interfere with IgM but not with IgA binding. Furthermore, it has already been shown that deglycosylation of human SC allows it to inhibit binding of biotinylated native SC to pIgA with 10 times greater efficiency than native SC itself (50), suggesting that some of the carbohydrate moieties on human pIgR may actually impede binding even of pIgA.”

Accordingly, in some embodiments, a deglycosylated variant of the recombinant pIgR including R/HpIgR is used to improve binding affinity to dIgA. In some embodiments, this may be achieved by expressing the pIgR in a glycan-deficient cell line known in the art such as, for example, a glycan deficient CHO cell line.

In plural embodiments, recombinant pIgR comprises a deletion in the transmembrane domain (ΔTM) to allow for convenient secretion of the recombinant protein and ease of use as a diagnostic/prognostic/screening agent.

In some embodiments, the recombinant pIgR comprises a heterologous detection domain.

Multiple detection domains are known in the art and are encompassed.

In some embodiments, the recombinant pIgR comprises a heterologous binding domain.

Multiple binding domains are known in the art and are encompassed.

In other embodiments, the recombinant pIgR is bound to a solid support. Solid supports include plates, wells, beads, agarose particles, nitrocellulose strips, etc.

In some embodiments, recombinant pIgR is produced in glycan deficient cells such as glycan deficient CHO cells to enhance preferential binding to dIgA over IgM.

In some embodiments, the recombinant pIgR is derived from a primate such as human pIgR and comprises at least one immunoglobulin-like domain derived from a non-primate such as rabbit, mouse, rat.

In some embodiments, the recombinant pIgR comprises an amino acid sequence set out in SEQ ID NO:2, or SEQ ID NO: 4, or SEQ ID NO: 6, or SEQ ID NO: 12, or SEQ ID NO: 14, or SEQ ID NO: 16, or an dIgA-binding part thereof or and a dIgA binding variant thereof. Illustrative variants comprise at least 70% amino acid sequence identity to one of SEQ ID NO: 2, 4, 6, 12, 14 or 16 or deletion variants thereof lacking a cytoplasmic domain.

Variants include deletion, substitution and insertional variants. Illustrated herein are human derived pIgR varied by one or more immunoglobulin domains (D). Variants include “parts” which includes fragments comprising from about 50%, 60%, 70%, 80%, 85%, 90%, 95% of the reference sequence. Substitution for an equivalent domain from a lower mammal such as a rat, mouse or rabbit domain.

“Variants” of the recited amino acid sequences are also contemplated. Variant molecules are designed to retain the dIgA binding functional activity of the pre-modified recombinant pIgR or to exhibit enhanced activity. Polypeptide variants according to the invention can be identified either rationally, or via established methods of mutagenesis (see, for example, Watson, J. D. et al., “Molecular Biology of the Gene”, Fourth Edition, Benjamin/Cummings, Menlo Park, California, 1987). Random mutagenesis approaches require no a priori information about the sequence that is to be mutated. This approach has the advantage that it assesses the desirability of a particular mutant based on its function, and thus does not require an understanding of how or why the resultant mutant protein has adopted a particular conformation. Indeed, the random mutation of target gene sequences has been one approach used to obtain mutant proteins having desired characteristics (Leatherbarrow R., J. Prot. Eng., 1:7-16, 1986; Knowles J. R., Science, 236:1252-1258, 1987; Shaw W. V., Biochem. J., 246:1-17, 1987; Gerit J. A., Chem. Rev., 87:1079-1105, 1987). Alternatively, where a particular sequence alteration is desired, methods of site-directed mutagenesis can be employed. Thus, such methods may be used to selectively alter only those amino acids of the protein that are believed to be important (Craik C. S., Science, 228:291-297, 1985; Cronin et al., Biochem., 27: 4572-4579, 1988; Wilks et al., Science, 242:1541-1544, 1988). Illustrative amino acids affect glycosylation of the recombinant pIgR. Polypeptides, resulting from rational or established methods of mutagenesis or from combinatorial chemistries, may comprise conservative amino acid substitutions. It is well understood in the art that some amino acids may be changed to others with broadly similar properties without changing the nature of the activity of the polypeptide (conservative substitutions, see Table 3).

Variant pIgR polypeptides comprises at least 50% sequence identity to herein amino acid sequence at least over the immunoglobulin-like domain region.

The terms and “sequence identity” as used herein refer to the extent that sequences are identical or functionally or structurally similar on an amino acid-by-amino acid basis over a window of comparison. Thus, a “percentage of sequence identity”, for example, is calculated by comparing two optimally aligned sequences over the window of comparison, determining the number of positions at which the identical amino acid residue (e.g. Ala, Pro, Ser, Thr, Gly, Val, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” will be understood to mean the “match percentage” calculated by the DNASIS computer program (Version 2.5 for windows; available from Hitachi Software engineering Co., Ltd., South San Francisco, California, USA) using standard defaults as used in the reference manual accompanying the software. Similar comments apply in relation to sequence similarity which counts as identical, substitutions involving conservative substitutions.

Preferably, the percentage similarity between a particular sequence and a reference sequence (nucleotide or amino acid) is at least about 60% or at least about 70% or at least about 80% or at least about 90% or at least about 95% or above such as at least about 96%, 97%, 98%, 99% or greater. Percentage similarities or identities between 60% and 100% are also contemplated such as 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In another embodiment there is provided recombinant pIgR encoded by the sequence of nucleotides set out in SEQ ID NO:1, SEQ ID NO:3, SEQ ID NO:5, SEQ ID NO:11, SEQ ID NO:13 or SEQ ID NO:15, or a dIgA-binding and optionally IgM-non binding variant thereof having at least 60% nucleotide sequence identity thereto or at least 60% nucleotide sequence identity to deletion variants thereof lacking a cytoplasmic domain.

In some convenient embodiments, the recombinant pIgR is a human recombinant pIgR variant comprising at least one immunoglobulin-like domain derived from a rabbit.

In some embodiments, (human) pIgR that binds pIgM and dIgA is employed. In some embodiments, IgG and/or IgM, are depleted using known protocols, and, if IgM is depleted then the human pIgR is selective for dIgA among what is left in the sample. In some embodiments, for diagnosis of many infections it may be preferable to detect both dIgA and pIgM—certainly IgA plus IgM is reported in the literature for hepatitis E and as illustrated herein there are some samples for hepatitis A or hepatitis E in which either IgM or dIgA are much stronger, suggesting that their combined detection is useful. While this could be achieved using a mixture, for example, of anti-IgM and the R/HpIgR, it may more conveniently be achieved with a mixture of HpIgR and R/HpIgR (so that the IgM does not outcompete the dIgA), or with HpIgR alone if it is present in excess over the sum of IgM and dIgA. It is proposed to be preferable to use two variations of the same reagent rather than one antibody plus a recombinant protein. In particular, pIgR is more thermally stable than antibody which is an advantage for test production.

The use of recombinant pIgR is also highly advantageous not least because the reagent displays low background (at least 50% less background compared to antibody based reagents) in binding assays unlike most antibody based binding agents. In addition, recombinant pIgR displays high thermal stability. For example, lyophilised recombinant pIgR retained 50% activity at 60° C. and 100% activity at 45° C. after three weeks prior to reconstitution, which compares favourably to the rapid loss of activity for dried anti-IgM antibody under the same conditions.

In embodiments, where substantially only dIgA (or IgM) is to be detected, recombinant human pIgR or dIgA and IgM binding variants may be employed as the binding reagent, but specifically detection of bound dIgA (either dIgA1 or dIgA2) is achieved using anti-IgA1/IgA2, in this embodiment, the presence of IgM is not a problem.

In some embodiments the process is for use in a method of assessing conditions or infections of a mucosal surface or associated tissues, or immunity thereto. Illustrative applications to specific antigens are described in the Figures and Figure legends for FIGS. 10 to 22 inclusive and these general protocols are expressly contemplated herein as well as routine variations thereto.

Illustrative mucosal surfaces include the upper and lower respiratory tracts, the gut and gut-associated lymphoid tissue, the genital tract and the liver.

Illustrative infections include without limitation those mediated by bacteria, viruses, parasites and other infectious organisms. In certain embodiments, infectious agents include, HIV, leprosy, syphilis, hepatitis (e.g., HEV, HAV, HCV) dengue virus, measles, rubella etc.

Illustrative conditions include diseases of organs such as the respiratory tract, lungs, gut, genital tract and liver. Gut conditions include ulcerative colitis, Crohn's disease, IBS, leaky gut syndrome etc.

Reference to “subject” includes humans and a wide range of mammalian or other animals including wild and domesticated animals, pets, pests and potential vehicles for emerging infectious diseases. In relation to subjects, these may have an infection, they may have had exposure to infection or they have had exposure to an infectious agent.

In one embodiment, FIG. 10 is a schematic of one preferred experimental approach for detecting the presence of antigen-specific dIgA in a sample such as human serum or plasma. Recombinant R/HpIgR-cyto is immobilised on the ELISA plate, and incubated with serum or other samples. Dimeric IgA is captured on the solid phase, and after washing to remove other sample components (such as IgA and IgG that are not captured, left), the presence of antigen-specific dIgA is detected by sequential addition of antigen that is, for example, either biotinylated, or reacted with a biotinylated monoclonal antibody against the antigen, and streptavidin-HRP. In this way, any antigen that is immobilised by reaction with antigen-specific dIgA will give a signal through the biotin-streptavidin interaction or an equivalent reagent.

In one embodiment, FIG. 11 is a schematic of one preferred experimental approach for detecting the presence of hepatitis A virus-specific dIgA in serum (right), compared to detection of HAY-specific IgM using the standard method of anti-IgM capture (left).

In one embodiment, FIG. 16 is a schematic of a second preferred method for detection of antigen-specific dIgA (or IgM), in which antigen is coated directly onto the ELISA plate (in this case, hepatitis E virus (HEV) antigen). Serum samples are applied to the plate and antigen-specific antibodies, including IgM and dIgA, bind to the antigens and are then detected with either anti-IgM HRP, or R/HpIgR and anti-human SC HRP. After final washing, signal is generated with TMB substrate or equivalent reagent.

In another embodiment, FIG. 23 provides a model outlining the pathogenic consequences of acute human immunodeficiency (HIV) infection, which leads to rapid CD4 depletion in the gut as well as the periphery, with a subsequent reduction in gut barrier function, increased leakage of gut contents and microbial translocation, leading to increased immune activation which drives pathogenesis and further reduction of CD4 T-cell levels (Brenchley et al, Nat Med 12: 1365-1371, 2006). There is an unmet need for simple, standardised assays that can detect one or more of these steps in the pathogenesis pathway so that appropriate interventions can be provided to patients, with only CD4 testing having been integrated into the standard of care for HIV-infected patients. Detection of CD4 depletion in the gut requires endoscopy; detection of decreased gut barrier function requires complicated sugar challenge studies or other methods; detection of gut leakage and microbial translocation can be achieved using markers such as bacterial LPS or 16sRNA in serum but results are highly variable due in part to the wide variation in gut microbiota between individuals; immune activation requires complex Flow cytometry protocols that are difficult to standardise across instruments/operators.

FIG. 24 provides a schematic illustration of the increase in microbial translocation due to gut leakage induced by pathogenic HIV or SIV infection, compared to normal low levels of translocation in nonpathogenic SIV infection.

FIG. 25 illustrates that one expected consequence of increased microbial translocation is the induction of increased IgA responses due to mucosal antigen exposure. M. French et al Journal of Infectious Diseases 200; 2009 demonstrated that indeed the total level of IgA in HIV patients after 6 years of follow up was inversely correlated with the level of CD4 T-cells in patients undergoing highly active antiretroviral therapy, suggesting that even in patients being treated with the most effective current antiviral therapies, microbial translocation contributes to pathogenesis. However these results also show that total IgA is highly variable between individuals, and does not provide a prognostic marker that can be used in management of individual patients.

FIG. 26 provides a schematic of the increase in microbial translocation due to gut leakage induced by pathogenic HIV or SIV infection, compared to normal low levels of translocation in nonpathogenic SIV infection, showing the expected effect on dimeric IgA and secretory IgA levels in the plasma compartment. Under normal conditions or nonpathogenic SIV infection, gut barrier integrity is maintained and the level of SIgA in the lumen of the gut reflects the amount of its precursor dIgA in the lamina propria. Only a minimal amount of SIgA is returned to the plasma compartment, either through active transport by M-cells in the gut, or a small amount of gut leakage. The amount of leakage or active transport can be estimated by comparing the serum/plasma concentration of SIgA to that of its precursor dIgA, giving a ratio of SIgA/dIgA. Under conditions of pathogenic HIV or SIV infection, or other physiological challenges that result in gut leakage, the total amount of dIgA is likely to be somewhat elevated and may lead to higher levels of SIgA secretion into the lumen. However a much higher proportion of SIgA will be returned to the plasma compartment due to passive leakage through the compromised gut barrier, resulting in an elevated SIgA/dIgA ratio.

In another embodiment, the present specification provides a process for assessing gut wall integrity. In some embodiments, and as discussed herein, this assessment provides a prognostic marker for HIV infected patients. In some embodiments, the process comprises determining the relative levels or a ratio of SIgA and dIgA in a sample from a human subject. In some embodiments, the process comprises the step of (i) contacting a biological sample with recombinant polymeric immunoglobulin (Ig) receptor (pIgR) or an dIgA-binding variant thereof, wherein the pIgR or variant binds dIgA and IgM; or wherein the pIgR or variant binds dIgA and substantially fails to bind IgM, and wherein the pIgR substantially does not bind monomeric IgA or secretory IgA and step (ii) determining the level or presence of dIgA that has bound to pIgR. In some embodiments, step (ii) comprises detecting a complex between dIgA and pIgR or a complex between bound dIgA and an antigen essentially as described herein. In another expression of this embodiment, the specification contemplates a process of determining the dIgA/SIgA ratio for use in assessing HIV infected subjects.

Secretory IgA (SIgA) is normally found in only trace amounts in plasma, but in patients with compromised gut barrier integrity, it is proposed herein that a larger proportion of SIgA will leak across the gut barrier and enter the plasma. The normal concentration of SIgA will differ between individual patients, because patients have widely varying levels of the SIgA precursor, dIgA. It is therefore useful to simultaneously measure the concentration of both dIgA and SIgA, with the ratio of SIgA to dIgA in the individual patient providing a measure of gut integrity/leakage.

FIG. 27 provides a schematic of one of several typical assays that can be used to measure the relative amount of different IgA forms in order to estimate the SIgA/dIgA ratio. In this example, the amount of dIgA is measured by capture of dIgA using R/HpIgR, and detection using monoclonal antibodies against either IgA1, or IgA2, or against both IgA subclasses. Monomeric IgA does not bind to pIgR; SIgA does bind to R/HpIgR but with lower affinity than dIgA and can be removed by washing with 3.5 M urea if desired. SIgA is measured in the same way but using anti-SC antibody capture instead of R/HpIgR. The SIgA/dIgA ratio is then calculated as a simple ratio of the assay reactivities for SIgA and dIgA.

As shown in the Examples, a proportion of patients infected with HIV exhibit an elevated level of SIgA compared to dIgA in patient plasma, consistent with elevated immune activation markers in these patients. In further embodiments, gut leakage can be determined by examining the individual IgA isotypes, because IgA2 (dIgA2, SIgA2) represents around 50% of IgA produced in the gut mucosa, but only around 10% of IgA in other tissues, and thus the ratio of SIgA2 to dIgA2 is likely to provide a very sensitive measurement of gut leakage in an individual patient.

Accordingly, in some embodiments, the process comprises measuring the levels of SIgA2 and dIgA2 and determining the ratio of SIgA2 to dIgA2 wherein the ratio relative to a control is indicative of the presence or absence or degree of gut leakage. In some embodiments, this ratio provides marker for HIV infected patients thereby potentially facilitating improved management of HIV infection in a subject. These assays are simple and have potential for high throughput as potential for incorporation into quantitative point of care devices.

Because the reaction between pIgR and dIgA is highly conserved across not only mammals but all vertebrate species, it is proposed that this process will have utility for detection of dIgA, and IgM when desirable, in a wide variety of species, providing a convenient and universal reagent for detection of immune activation or dIgA and/or IgM responses in species such as bats and other wild or domesticated animals for which there may be no available anti-immunoglobulin reagents. This will be useful in diagnosis of diseases of agricultural interest in domesticated animals, and for the diagnosis of disease and detection of host reservoir species for emerging infectious diseases.

The present invention enables the use of the specific interaction between the recombinant expressed forms of pIgR and dIgA (plus or minus IgM), allowing pIgR to be used for specific binding or detection of dIgA (plus or minus IgM) to solid surfaces or other assay components as desired.

In an embodiment, the herein described process and/or recombinant pIgR or variants thereof as described herein are sub-licensed for use in antigen screening or antibody selection and purification.

Treatment protocols are contemplated based upon the results of diagnosis or prognosis testing as described herein.

In some embodiments, the process further comprises: (a) generating data using a process as described herein; (b) transforming the data into computer-readable form; and (c) operating a computer to execute an algorithm, wherein the algorithm determines closeness-of-fit between the computer-readable data and data indicating a diagnosis of a disease or condition. In some embodiments, the algorithm comprises an artificial intelligence program, such as a fuzzy logic, cluster analysis or neural network. The subject methods may also be used in a personalized or a population medicine approach in the management of pathology platforms.

The present disclosure provides a computer program and hardware for diagnosis in a subject once off, over time or in response to treatment or other affectors. Values are assigned to complex levels which are stored in a machine readable storage medium. A computer program product is one able to convert such values to code and store the code in a computer readable medium and optionally capable of assessing the relationship between the stored data and incoming data and optionally a knowledge database to assess a potential TB status and/or pneumonia.

The present specification therefore provides a web-based system where data on levels of complex are provided by a client server to a central processor which analyses and compares to a control and optionally considers other information such as patient age, sex, weight and other medical conditions and then provides a diagnostic report.

The assay may, therefore, be in the form of a kit or computer-based system which comprises the reagents necessary to form and detect the herein described antibody complexes and the computer hardware and/or software including an algorithm to facilitate determination and transmission of reports to a clinician.

The present invention contemplates a method of allowing a user to determine the status of a subject with respect to TB, the method including:
(a) receiving data from the conduct of the process as herein described from the user via a communications network;
(b) processing the subject data via multivariate analysis to provide a diagnostic index value;
(c) determining the status of the subject in accordance with the index value in comparison with predetermined values; and
(d) transferring an indication of the status of the subject to the user via the communications network.

Conveniently, the method generally further includes:
(a) having the user determine the data using a remote end station; and
(b) transferring the data from the end station to the base station via the communications network.

As used herein, the term “binds specifically,” and the like when referring to an antigen-binding molecule refers to a binding reaction which is determinative of the presence of an antigen in the presence of a heterogeneous population of proteins and other biologics. Thus, under designated immunoassay conditions, the specified antigen-binding molecules bind to a particular antigen and do not bind in a significant amount to other proteins or antigens present in the sample. Specific binding to an antigen under such conditions may require an antigen-binding molecule that is selected for its specificity for a particular antigen. For example, antigen-binding molecules can be raised to a selected protein antigen, which bind to that antigen but not to other proteins present in a sample. A variety of immunoassay formats may be used to select antigen-binding molecules specifically immuno-interactive with a particular protein. For example, solid-phase ELISA immunoassays are routinely used to select monoclonal antibodies specifically immuno-interactive with a protein. See Harlow and Lane (1988) Antibodies, A Laboratory Manual, Cold Spring Harbor Publications, New York, for a description of immunoassay formats and conditions that can be used to determine specific immunoreactivity.

For example, specific recognition is provided by a primary antibody (polyclonal or monoclonal) and a secondary detection system is used to detect presence (or binding) of the primary antibody. Detectable labels can be conjugated to the secondary antibody, such as a fluorescent label, a radiolabel, or an enzyme (e.g., alkaline phosphatase, horseradish peroxidase) which produces a quantifiable, e.g., colored, product. In another suitable method, the primary antibody itself can be detectably labeled. For example, a protein-specific monoclonal antibody, can be used both as an immunoadsorbent and as an enzyme-labeled probe to detect and quantify complexes formed in the present process or kit.

The amount of such protein present in a sample can be calculated by reference to the amount present in a standard or reference preparation using a linear regression computer algorithm (see Lacobilli et al., (1988) Breast Cancer Research and Treatment 11:19-30). In other embodiments, two different monoclonal antibodies to the protein of interest can be employed, one as the immunoadsorbent and the other as an enzyme-labeled probe.

Assays illustrated in the Examples are done in ELISA format with a single antigen per well, per single antibody form or class or isotype. However there are well known methods where they could be combined into a single assay for example using Luminex beads or similar where multiple individual antigens are coated on beads having different intensity of fluorescent label that can be discriminated in an instrument, and the amount of antibody binding to antigen on each bead can be separately measured from the single sample. Similarly the Luminex beads can be coated with antibody or other reagents to capture the individual antibody forms or isotypes from a sample, and then labelled antigen (or antigens) is added and the different isotype reactivities are assessed. The same can be done in micro-arrays or other arrays. Having established useful parameters in ELISA, it is then routine to transfer these findings to multiplex formats. In lateral flow and other point of care devices, where the sample flows across a membrane, it is easy to have the separate antigens present on the membrane as separate stripes or spots, and then detect the antibodies of one or more isotypes together; or else have different capture antibodies for the antibody isotopes, and then detect the (labelled) antigen binding to each of the immobilized antibody stripes or spots. The latter method (isotype capture, detection of labelled antigen bound by the immobilised patient antibody) is a most preferred approach.

Additionally, recent developments in the field of protein capture arrays permit the simultaneous detection and/or quantification of a large number of proteins. For example, low-density protein arrays on filter membranes, such as the universal protein array system (Ge (2000) Nucleic Acids Res. 28(2):e3) allow imaging of arrayed antigens using standard ELISA techniques and a scanning charge-coupled device (CCD) detector. Immuno-sensor arrays have also been developed that enable the simultaneous detection of clinical analytes. It is now possible using protein arrays, to profile protein expression in bodily fluids, such as in sera of healthy or diseased subjects, as well as in subjects pre- and post-drug treatment.

Protein capture arrays typically comprise a plurality of protein-capture agents each of which defines a spatially distinct feature of the array. The protein-capture agent can be any molecule or complex of molecules which has the ability to bind a protein and immobilize it to the site of the protein-capture agent on the array. The protein-capture agent may be a protein whose natural function in a cell is to specifically bind another protein, such as an antibody or a receptor. Alternatively, the protein-capture agent may instead be a partially or wholly synthetic or recombinant protein which specifically binds a protein. Alternatively, the protein-capture agent may be a protein which has been selected in vitro from a mutagenized, randomized, or completely random and synthetic library by its binding affinity to a specific protein or peptide target. The selection method used may optionally have been a display method such as ribosome display or phage display, as known in the art. Alternatively, the protein-capture agent obtained via in vitro selection may be a DNA or RNA aptamer which specifically binds a protein target (see, e.g., Potyrailo et al., (1998) Anal. Chem. 70:3419-3425; Cohen et al. (1998) Proc. Natl. Acad. Sci. USA 95:14272-14277; Fukuda, et al. (1997) Nucleic Acids Symp. Ser. 37:237-238; available from SomaLogic). For example, aptamers are selected from libraries of oligonucleotides by the Selex™ process and their interaction with protein can be enhanced by covalent attachment, through incorporation of brominated deoxyuridine and UV-activated crosslinking (photoaptamers). Aptamers have the advantages of ease of production by automated oligonucleotide synthesis and the stability and robustness of DNA; universal fluorescent protein stains can be used to detect binding. Alternatively, the in vitro selected protein-capture agent may be a polypeptide (e.g., an antigen) (see, e.g., Roberts and Szostak (1997) Proc. Natl. Acad. Sci. USA 94:12297-12302).

An alternative to an array of capture molecules is one made through ‘molecular imprinting’ technology, in which peptides (e.g., from the C-terminal regions of proteins) are used as templates to generate structurally complementary, sequence-specific cavities in a polymerisable matrix; the cavities can then specifically capture (denatured) proteins which have the appropriate primary amino acid sequence (e.g., available from ProteinPrint™ and Aspira Biosystems).

Exemplary protein capture arrays include arrays comprising spatially addressed TB antigens or antibody binding agents, which can facilitate extensive parallel analysis of numerous antigens and antibodies. Such arrays have been shown to have the required properties of specificity and acceptable background, and some are available commercially (e.g., BD Biosciences, Clontech, BioRad and Sigma). Various methods for the preparation of arrays have been reported (see, e.g., Lopez et al. (2003) 1 Chromatogr. B 787:19-27; Cahill (2000) Trends in Biotechnology 7:47-51; U.S. Pat. App. Pub. 2002/0055186; U.S. Pat. App. Pub. 2003/0003599; PCT publication WO 03/062444; PCT publication WO 03/077851; PCT publication WO 02/59601; PCT publication WO 02/39120; PCT publication WO 01/79849; PCT publication WO 99/39210).

Immunoglobulin antigen-binding molecules are made either by conventional immunization (e.g., polyclonal sera and hybridomas), or as recombinant fragments, usually expressed in E. coli, after selection from phage display or ribosome display libraries (e.g., available from Cambridge Antibody Technology, BioInvent, Affitech and Biosite). Alternatively, ‘combibodies’ comprising non-covalent associations of VH and VL domains, can be produced in a matrix format created from combinations of diabody-producing bacterial clones (e.g., available from Domantis). Exemplary antigen-binding molecules for use as protein-capture agents include monoclonal antibodies, polyclonal antibodies, Fv, Fab, Fab′ and F(ab′)₂ immunoglobulin fragments, synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv, single domains from camelids or engineered human equivalents.

Individual spatially distinct protein-capture agents are typically attached to a support surface, which is generally planar or contoured. Common physical supports include glass slides, silicon, microwells, nitrocellulose or PVDF membranes, and magnetic and other microbeads.

While microdrops of protein delivered onto planar surfaces are widely used, related alternative architectures include CD centrifugation devices based on developments in microfluidics (e.g., available from Gyros) and specialized chip designs, such as engineered microchannels in a plate (e.g., The Living Chip™, available from Biotrove) and tiny 3D posts on a silicon surface (e.g., available from Zyomyx).

Particles in suspension can also be used as the basis of arrays, providing they are coded for identification; systems include color coding for microbeads (e.g., available from Luminex, Bio-Rad and Nanomics Biosystems) and semiconductor nanocrystals (e.g., QDots™, available from Quantum Dots), and barcoding for beads (UltraPlex™, available from Smartbeads) and multimetal microrods (Nanobarcodes™ particles, available from Surromed). Beads can also be assembled into planar arrays on semiconductor chips (e.g., available from LEAPS technology and BioArray Solutions). Where particles are used, individual protein-capture agents are typically attached to an individual particle to provide the spatial definition or separation of the array. The particles may then be assayed separately, but in parallel, in a compartmentalized way, for example in the wells of a microtiter plate or in separate test tubes.

In operation, a protein sample (see, e.g., U.S. Pat. App. Pub. 2002/0055186), is delivered to a protein-capture array under conditions suitable for protein or peptide binding, and the array is washed to remove unbound or non-specifically bound components of the sample from the array. Next, the presence or amount of protein or peptide bound to each feature of the array is detected using a suitable detection system. The amount of protein bound to a feature of the array may be determined relative to the amount of a second protein bound to a second feature of the array. In certain embodiments, the amount of the second or subsequent protein in the sample is already known or known to be invariant.

In an illustrative example, fluorescence labeling can be used for detecting protein bound to the array. The same instrumentation as used for reading DNA microarrays is applicable to protein-capture arrays. For differential display, capture arrays (e.g. antibody arrays) can be probed with fluorescently labeled proteins from or are labeled with different fluorophores (e.g., Cy-3 and Cy-5) and mixed, such that the color acts as a readout for changes in target abundance. Fluorescent readout sensitivity can be amplified 10-100 fold by tyramide signal amplification (TSA) (e.g., available from PerkinElmer Lifesciences). Planar waveguide technology (e.g., available from Zeptosens) enables ultrasensitive fluorescence detection, with the additional advantage of no washing procedures. High sensitivity can also be achieved with suspension beads and particles, using phycoerythrin as label (e.g., available from Luminex) or the properties of semiconductor nanocrystals (e.g., available from Quantum Dot). Fluorescence resonance energy transfer has been adapted to detect binding of unlabelled ligands, which may be useful on arrays (e.g., available from Affibody). Several alternative readouts have been developed, including adaptations of surface plasmon resonance (e.g., available from HTS Biosystems and Intrinsic Bioprobes), rolling circle DNA amplification (e.g., available from Molecular Staging), mass spectrometry (e.g., available from Sense Proteomic, Ciphergen, Intrinsic and Bioprobes), resonance light scattering (e.g., available from Genicon Sciences) and atomic force microscopy (e.g., available from BioForce Laboratories). A microfluidics system for automated sample incubation with arrays on glass slides and washing has been co-developed by NextGen and Perkin Elmer Life Sciences.

In certain embodiments, the techniques used for detection of dIgA or other pre-selected products will include internal or external standards to permit quantitative or semi-quantitative determination of those products, to thereby enable a valid comparison of the level or functional activity of these expression products in a biological sample with the corresponding expression products in a reference sample or samples. Such standards can be determined by the skilled practitioner using standard protocols. In specific examples, absolute values for the level or functional activity of individual expression products are determined. Controls may include¬individual and population control and samples from diagnostic tests—an earlier time point.

In specific embodiments, the diagnostic method is implemented using a system as disclosed, for example, in International Publication No. WO 02/090579 and in copending PCT Application No. PCT/AU03/01517 filed Nov. 14, 2003, comprising at least one end station coupled to a base station. The base station is typically coupled to one or more databases comprising predetermined data from a number of individuals representing the level TB antigen specific antibodies and their isotype structure (dimeric/polymeric) or subclass, when the predetermined data was collected. In operation, the base station is adapted to receive from the end station, typically via a communications network, subject data representing a measured or normalized level of at least one antibody type in a biological sample obtained from a test subject and to compare the subject data to the predetermined data stored in the database(s). Comparing the subject and predetermined data allows the base station to determine the status of the subject in accordance with the results of the comparison. Thus, the base station attempts to identify individuals having similar parameter values to the test subject and once the status has been determined on the basis of that identification, the base station provides an indication of the diagnosis to the end station. In an embodiment, recombinant pIgR is sub-licensed for use in TB antigen screening or TB serological diagnosis.

Each embodiment in this specification is to be applied mutatis mutandis to every other embodiment unless expressly stated otherwise.

Functionally equivalent methods and kits employing such methods are clearly within the scope of the invention as described herein.

The present invention is further described by the following non-limiting Examples.

### Example 1

ELISA Shows Preferential Binding of Chimeric pIgR to dIgA Over IgM, but Strong Binding of Human pIgR to IgM

As shown in FIG. 8, an ELISA is performed comparing the binding of HpIgR and R/HpIgR to human IgM and dIgA indicating preferential binding of Chimeric pIgR to dIgA over IgM, but strong binding of human pIgR to IgM. HpIgR or R/HpIgR were immobilised on 96-well Nunc Immulon plates overnight at 4° C. Dilutions of purified human IgM or dIgA in PBS were bound to the immobilised pIgR forms overnight. After washing, the captured IgM or dIgA were detected using anti-IgM or anti-IgA conjugated to horseradish peroxidase (HRP) and colorimetric substrate TMB. The results demonstrate that HpIgR shows preferential binding to IgM (magenta) as well as binding to dIgA (green), whereas R/HpIgR shows greatly reduced binding to IgM (yellow) but retains strong binding to dIgA (blue).

### Example 2

ELISA Shows Detection of Immobilised dIgA Using R/HpIgR With Negligible Background

An ELISA was performed comparing the detection of immobilised dIgA using R/HpIgR. Dilutions of purified dIgA or no dIgA (mock) were immobilised on 96-well Nunc Immulon plates that were previously coated with anti-IgA, so that the dIgA was bound to the plate by antibody-antigen interaction rather than passive absorption. The dIgA was detected using R/HpIgR (“tailless”) or no pIgR (“mock”), anti-secretory component and anti-mouse HRP and TMB substrate. The results (see FIG. 9) demonstrate R/HpIgR is able to detect dIgA at the lowest concentration tested (31 ng/ml) with strong signal in ELISA with negligible background.

### Example 3

### ELISA Showing Effective Depletion of HAV-Specific IgM in IgM Capture

An ELISA was conducted demonstrating the detection of HAV-specific IgM in IgM capture, using serial dilutions of serum from a patient with acute HAV infection (Accurun HAV panel sample 121). The serum sample is either untouched before dilution (untouched, purple) or substantially depleted of IgM using Capture-Select IgM (BAC) (red). The results (see FIG. 12) show that this IgM depletion method reduces the level of HAY-specific IgM in the sample by around 256-fold compared to untouched serum.

### Example 4

ELISA Shows Detection of Hepatitis A Virus-Specific dIgA Compared to IgM in an Individual Patient, With or Without Depletion of IgM

An ELISA was conducted demonstrating the detection of HAV-specific dIgA in R/HpIgR capture, using serial dilutions of serum from a patient with acute HAV infection (Accurun HAV panel sample 121). The serum sample is either untouched before dilution (untouched, purple) or substantially depleted of IgM using Capture-Select IgM (BAC) (red). The results show firstly the strong signal that is obtained demonstrating the detection of HAV-specific dIgA, and secondly that this signal is specific for dIgA not IgM because the IgM depletion method did not substantially reduce the level of HAV-specific reactivity compared to untouched serum, in contrast to the results shown in FIG. 12 for IgM detection.

### Example 5

R/HpIgR Capture of Antigen-Specific dIgA for the Diagnosis of Acute HAV Infection

An ELISA was conducted demonstrating the detection of HAY-specific dIgA in R/HpIgR capture, using serial dilutions of serum from a patient with acute HAV infection (Accurun HAV panel sample 121). The serum sample is either untouched before dilution (untouched, purple) or substantially depleted of IgM using Capture-Select IgM (BAC) (red). The results (see FIG. 13) show firstly the strong signal that is obtained demonstrating the detection of HAV-specific dIgA, and secondly that this signal is specific for dIgA not IgM because the IgM depletion method did not substantially reduce the level of HAY-specific reactivity compared to untouched serum, in contrast to the results shown in FIG. 12 for IgM detection.

An ELISA was performed demonstrating the detection of HAV-specific dIgA in R/HpIgR capture, using sera from patients with or without acute HAV infection (Accurun HAV panel, positive (POS), low positive (LOW POS), or negative (NEG)). The results (FIG. 14) show the strong detection of HAY-specific dIgA in all POS samples and in one of two LOW POS samples, with minimal background reactivity in NEG samples, demonstrating the utility of R/HpIgR capture of antigen-specific dIgA for the diagnosis of acute HAV infection.

### Example 6

### R/HpIgR Capture and Utility of HpIgR Capture for Diagnosis of Acute HEY Infection

An ELISA was conducted demonstrating the detection of hepatitis E virus (HEV)-specific dIgA in R/HpIgR capture or HpIgR capture, using sera from patients with or without acute HEV infection. On the left of FIG. 15, the ELISA OD of individual samples is shown, demonstrating the utility of R/HpIgR capture of antigen-specific dIgA for the diagnosis of acute HEV infection, with lower but still significant utility of HpIgR capture for this purpose, and negligible background reactivity in either example. On the right of FIG. 15, the reactivity of serial dilutions of each serum sample is shown, confirming the utility of R/HpIgR capture and lower utility of HpIgR capture for diagnosis of acute HEV infection. It is likely that the lower utility of HpIgR capture in these examples is due to the much higher overall concentration of IgM in serum versus dIgA, resulting in only a low proportion of the IgM captured by HpIgR being specific for HEV.

### Example 7

Detection of Antigen-Specific dIgA or IgM Illustrated Using HEY Infected Patients

Comparison of HEV-specific dIgA versus HEV-specific IgM. Both methods are able to detect all HEY-infected patients with strong ELISA signals (FIG. 17), compared to extremely low background for control (HEV-negative) patients in the dIgA assay, and low background in the IgM assay. Notably, some samples show higher levels of dIgA compared to IgM (sample J13, J7), while others show higher levels of IgM compared to dIgA (J4, J11). This demonstrates that the dIgA and IgM responses in patients are independent, and suggests that a combination of both IgM and dIgA detection may be useful in some desirable assay formats. FIG. 17 illustrates that the dIgA assay involved essentially no background while the commercial IgM assay which is highly optimised gives low but detectable background.

Comparison of HEY-specific dIgA versus HEV-specific IgM is conducted using sera that are either untouched, or substantially depleted of IgM using Capture-Select IgM, and then serially diluted. The results (FIG. 18) confirm that the IgM assay is specific for IgM, because the reactivity is ablated by IgM depletion, whereas the dIgA assay is predominantly specific for dIgA and not IgM, because the reactivity is only slightly affected by IgM depletion.

Comparison of HEV-specific dIgA versus HEV-specific IgM is conducted using sera that are either untouched, or substantially depleted of IgM using Capture-Select IgM. The results (FIG. 19) confirm that the IgM assay is specific for IgM, because the reactivity is ablated by IgM depletion, whereas the dIgA assay is predominantly specific for dIgA and not IgM, because the reactivity is only slightly affected by IgM depletion.

Comparison of HEV-specific dIgA versus HEV-specific IgM is conducted using sera that are either untouched, or substantially depleted of IgM using Capture-Select IgM. The results (FIG. 20) confirm that the IgM assay is specific for IgM, because the reactivity is ablated by IgM depletion, whereas the dIgA assay is predominantly specific for dIgA and not IgM, because the reactivity is only slightly affected by IgM depletion. The reduction in dIgA activity following IgM depletion is statistically significant when using a paired T-test to compare samples before and after depletion, but is not significant when using a Mann-Whitney test to compare the overall sample sets before and after depletion.

### Example 8

R/HpIgR Capture and Detection of Mouse dIgA

An ELISA is conducted demonstrating that the R/HpIgR can be used in both capture (A) and detection (B) of mouse dimeric IgA, with negligible background reactivity to monomeric (human) IgA. FIG. 21A. Dilutions of purified mouse IgA monoclonal antibody 3H1 (anti-HAV) or purified monomeric human IgA were coated on plates and detected with R/HpIgR and anti-SC antibodies. B. R/HpIgR was coated on plates and dilutions of purified mouse IgA monoclonal antibody 3H1 or purified monomeric human IgA were allowed to bind overnight, then detected with anti-mouse IgA or anti-human IgA. The binding of IgA from diverse species to human or rabbit pIgR is known in the art, and this demonstrates that the novel pIgR strategy described herein has utility for diagnosis of infection in other species.

An ELISA is conducted demonstrating that the R/HpIgR is equally effective for detection of mouse dimeric IgA and human dimeric IgA, with negligible background reactivity to monomeric human IgA. FIG. 22A. Dilutions of purified mouse IgA monoclonal antibody 3H1 (anti-HAV) or purified dimeric or monomeric human IgA were coated on plates and detected with R/HpIgR and anti-SC antibodies.

### Example 9

### Assessment of HIV Pathogenesis and Illustration of Utility of SIgA/dIgA Ratios in HIV-Infected Subjects

A schematic of one of several typical assays (see FIG. 27) that can be used to measure the relative amount of different IgA forms in order to estimate the SIgA/dIgA ratio. In this example, the amount of dIgA is measured by capture of dIgA using R/HpIgR, and detection using monoclonal antibodies against either IgA1, or IgA2, or against both IgA subclasses. Monomeric IgA does not bind to pIgR; SIgA does bind to R/HpIgR but with lower affinity than dIgA and can be removed by washing with 3.5 M urea if desired. SIgA is measured in the same way but using anti-SC antibody capture instead of R/HpIgR. The SIgA/dIgA ratio is then calculated as a simple ratio of the assay reactivities for SIgA and dIgA.

An ELISA is conducted demonstrating the detection of highly elevated SIgA2/dIgA2 (S/d) ratios in a proportion of HIV-infected patients, compared to the majority of HIV-infected patients and all control subjects (magenta). The assay cutoff for elevated SIgA/dIgA was set as the mean plus 3 standard deviations of the SIgA/dIgA ratio among non-HIV control subjects, and 7/30 HIV-infected subjects showed SIgA/dIgA ratios above this cutoff. Notably, the range of SIgA/dIgA ratios among normal subjects is smaller than the range for SIgA or dIgA alone, because the role of dIgA as the precursor of SIgA provides a normalising effect for each patient.

An ELISA is conducted demonstrating the total amount of SIgA in patient and control sera (arbitrary units). The amount of SIgA2 in normal patients varies over an 11-fold range, but all normal controls fall within a cutoff of the mean plus 3 standard deviations. The amount of SIgA2 in HIV-infected patients varies over a slightly larger range (16-fold), but only 2/30 patients are above the cutoff range (see FIG. 29). Among the HIV-infected patients, those patients who demonstrated elevated SIgA2/dIgA2 ratios in FIG. 28 are indicated with red markers (diamonds at ranks 11, 16, 19, 23, 24, 28 and 30). It can be seen that these patients with elevated SIgA2/dIgA2 ratios are found throughout much of the normal range of the total SIgA2 signal, and cannot be distinguished from the normal controls on the basis of the total SIgA2 alone. This confirms the utility of using SIgA/dIgA ratios because the role of dIgA as the precursor of SIgA provides a normalising effect for each patient. The R/HpIgR system provides the utility for measuring this ratio.

FIG. 30 illustrates a correlation of SIgA2/dIgA2 ratio versus the immune activation marker, CD8+HLA−DR+CD38+ T-cells, in a different HIV-infected population to that shown in FIGS. 28 and 29. While the overall correlation is low, it is apparent that patients with SIgA2/dIgA2 ratios of >4 in this experiment have elevated levels of immune activation markers (p<0.0001).

FIG. 31 illustrates a correlation of SIgA1/dIgA1 ratio versus the immune activation marker, CD8+HLA−DR+CD38+ T-cells, in the same population as FIG. 30. While the overall correlation is lower again than for IgA2, it is apparent that patients with SIgA1/dIgA1 ratios of >10 in this experiment have elevated levels of immune activation markers (p<0.015). The lower correlation for IgA 1 and higher cutoff ratio (10 versus 4) for significance highlights the value of specifically measuring IgA2 because of its predominant site of synthesis in the gut, being the tissue in which leakage of SIgA is likely to be clinically relevant marker of gut leakage and immune activation.

FIG. 32 illustrates a correlation of SIgA1/dIgA1 ratio versus SIgA2/dIgA2 ratio, in the same population as FIGS. 30 and 31. While SIgA1/dIgA1 ratios are significantly correlated with SIgA2/dIgA2, it is notable that there are some patients with highly elevated SIgA1/dIgA1 ratios and relatively low SIgA2/dIgA2 ratios. This suggests that there may be some value in measuring IgA1, or total IgA, in addition to IgA2 in the calculation of SIgA/dIgA ratios as a measure of gut leakage and immune activation.

The disclosure of every patent, patent application, and publication cited herein is hereby incorporated herein by reference in its entirety.

Many modifications will be apparent to those skilled in the art without departing from the scope of the present invention.

TABLE 1
Summary of sequence identifiers

| SEQUENCE ID NO: | DESCRIPTION |
|---|---|
| 1 | Nucleotide sequence of human pIgR |
| 2 | Amino acid sequence of human pIgR |
| 3 | Nucleotide sequence of rabbit pIgR |
| 4 | Amino acid sequence of rabbit pIgR |
| 5 | Nucleotide sequence of chimeric |
| | human/rabbit pIgR |
| 6 | Amino acid sequence of chimeric |
| | human/rabbit pIgR |
| 7 | Nucleotide sequence of N-terminal |
| | rabbit domain 1 chimeric |
| | human/rabbit pIgR |
| 8 | Amino acid sequence of N-terminal |
| | rabbit domain 1 chimeric |
| | human/rabbit pIgR |
| 9 | Nucleotide sequence of C-terminal |
| | human domains 2-4 of chimeric |
| | human/rabbit pIgR |
| 10 | Amino acid sequence of C-terminal |
| | human domains 2-4 of chimeric |
| | human/rabbit pIgR |
| 11 | Nucleotide sequence of human pIgR |
| | with cytoplasmic domain of CD4 |
| 12 | Amino acid sequence of human pIgR |
| | with cytoplasmic domain of CD4 |
| 13 | Nucleotide sequence of rabbit pIgR |
| | cytoplasmic domain with cytoplasmic |
| | domain of CD4 |
| 14 | Amino acid sequence of rabbit pIgR |
| | with cytoplasmic domain of CD4 |
| 15 | Nucleotide sequence of chimeric |
| | human/rabbit pIgR with |
| | cytoplasmic domain of CD4 |
| 16 | Amino acid sequence of chimeric |
| | human/rabbit pIgR with |
| | cytoplasmic domain of CD4 |
| 17 | Nucleotide sequence of N-terminal |
| | rabbit domain 1 of chimeric |
| | human/rabbit pIgR with |
| | cytoplasmic domain of CD4 |
| 18 | Amino acid sequence N-terminal |
| | rabbit domain 1 of chimeric |
| | human/rabbit pIgR with |
| | cytoplasmic domain of CD4 |
| 19 | Nucleotide sequence of C-terminal |
| | human domains 2-4 of chimeric |
| | human/rabbit pIgR with |
| | cytoplasmic domain of CD4 |
| 20 | Amino acid sequence of C-terminal |
| | human domains 2-4 of chimeric |
| | human/rabbit pIgR with |
| | cytoplasmic domain of CD4 |

TABLE 2
Amino acid sub-classification

| Sub-classes | Amino acids |
|---|---|
| Acidic | Aspartic acid, Glutamic acid |
| Basic | Noncyclic: Arginine, Lysine; |
| | Cyclic: Histidine |
| Charged | Aspartic acid, Glutamic acid, |
| | Arginine, Lysine, Histidine |
| Small | Glycine, Serine, Alanine, |
| | Threonine, Proline |
| Polar/neutral | Asparagine, Histidine, Glutamine, |
| | Cysteine, Serine, Threonine |
| Polar/large | Asparagine, Glutamine |
| Hydrophobic | Tyrosine, Valine, Isoleucine, |
| | Leucine, Methionine, Phenylalanine, |
| | Tryptophan |
| Aromatic | Tryptophan, Tyrosine, Phenylalanine |
| Residues that | Glycine and Proline |
| influence chain | |
| orientation | |

TABLE 3
Exemplary and Preferred Amino Acid Substitutions

| Original Residue | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn, His, Lys, | Asn |
| Glu | Asp, Lys | Asp |
| Gly | Pro | Pro |
| His | Asn, Gln, Lys, Arg | Arg |
| Ile | Leu, Val, Met, Ala, Phe, | Leu |
| | Norleu | |
| Leu | Norleu, Ile, Val, Met, Ala, | Ile |
| | Phe | |
| Lys | Arg, Gln, Asn | Arg |
| Met | Leu, Ile, Phe | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, | Leu |
| | Norleu | |

### BIBLIOGRAPHY

Brenchley et al. (2006) Nat Med 12(12): 1365-1371
Cahill (2000) Trends in Biotechnology 7:47-51
Cohen (2008) Science
Cohen et al. (1998) Proc. Natl. Acad. Sci. USA 95:14272-14277
Crago et al. (1984) J Immunol 132(1): 16-18
Craik C. S. (1985) Science, 228:291-297
Cronin et al., Biochem. (1988) 27: 4572-4579
French et al. (2009) J Infect Dis. 200(8): 1212-1215
Fukuda, et al. (1997) Nucleic Acids Symp. Ser. 37:237-238
Ge (2000) Nucleic Acids Res. 28(2):e3
Gerit J. A. (1987) Chem. Rev., 87:1079-1105
Harlow and Lane (1988) “Antibodies: A Laboratory Manual” Cold Spring Harbor Laboratory
He et al. (2007) Immunity 26(6): 812-826
Kaetzel and Bruno (2011) Mucosal immune defense: Immunoglobulin A
Knowles J. R. (1987) Science, 236:1252-1258
Lacobilli et al., (1988) Breast Cancer Research and Treatment 11:19-30
Leatherbarrow R. (1986) J. Prot. Eng., 1:7-16
Lopez et al. (2003) J. Chromatogr. B 787:19-27
Nilssen et al. (2004) Gut 53:487-493
Phillips-Quagliata et al. (2000) J Immunol 165: 2544-2555
Potyrailo et al., (1998) Anal. Chem. 70:3419-3425
Roberts and Szostak (1997) Proc. Natl. Acad. Sci. USA 94:12297-12302
Roe et al. (1999) J Immunol 162: 6046-52
Sambrook (2001) Molecular Cloning: A Laboratoy Manual, 3^{rd} Edition, CSHLP, CSH, NY
Shaw W. V. (1987) Biochem. J., 246:1-17
Watson, J. D. et al. (1987) “Molecular Biology of the Gene”, Fourth Edition, Benjamin/Cummings, Menlo Park, Calif.
Wild D. (2001) “The Immunoassay Handbook” Nature Publishing Group
Wilks et al. (1988) Science, 242:1541-1544
Woof and Russell (2011) Mucosal Immunology 4:590-597
Zhang et al. (2008) Nephrol Dial Transplant 23(1): 207-212

1-24. (canceled)

25. A kit for assessing immune status in a subject, the kit comprising:
(a) an immunographic device comprising a porous membrane operably connected to a sample portion, a test portion, and optionally a control portion; and further comprising a sucker portion, portion comprising a recombinant polymeric Ig Receptor (pIgR) comprising domain 1 that binds dimeric IgA antibody (dIgA) and substantially fails to bind IgM, and, optionally, a conjugate portion comprising one or more binding agents directed against IgA1 and/or IgA2; and
b) instructions for using the immunographic device to detect the presence of antigen specific dIgA antibody in the sample.

26. The kit of claim 25 wherein (a) further comprises a portion comprising an antigen of interest; and wherein b) instructions for using the immunographic device to detect the presence of specific dIgA antibody in the sample are for detecting the presence of antigen specific dIgA antibody.

27. The kit of claim 26 wherein the recombinant pIgR has the transmembrane domain and/or the cytoplasmic domain deleted.

28. The kit of claim 26 wherein the recombinant pIgR comprises a heterologous detection or binding domain.

29. The kit of claim 26 wherein the recombinant pIgR is recombinantly produced in a glycan deficient cell.

30. The kit of claim 26 wherein the recombinant pIgR is bound to a solid support.

31. The kit of claim 26 wherein the antigen of interest is an antigen of an infectious agent or an antigen associated with a condition of a subject that affects a mucosal surface or associated tissues.

32. The kit of claim 26 wherein the infectious agent is selected from HIV, leprosy, syphilis, hepatitis, dengue virus, measles and rubella.

33. The kit of claim 26 further comprising an anti-SIgA binding agent/antibody or anti-SC antibody, wherein the anti-SIgA binding agent/antibody or anti-SC antibody binds SIgA and forms an SIgA-binding agent/antibody complex.

34.-35. (canceled)

36. The kit of claim 26 wherein the biological sample is a blood, plasma, or serum sample.

37. The kit of claim 26 wherein the recombinant pIgR comprises rabbit, mouse, or rat pIgR domain 1.

38. The kit of claim 26 wherein the recombinant pIgR comprises rabbit pIgR domain 1.

39. The kit of claim 26 wherein the recombinant pIgR comprises human pIgR domains 2 to 5.

40. The kit of claim 32 where hepatitis is selected from: hepatitis A virus (HAV), hepatitis C virus (HCV) and hepatitis E virus (HEV).
